(19) United States
(12) Patent Application Publication
(10) Pub. No.: US 2009/0105245 A1
(43) Pub. Date: Apr. 23, 2009

(54) METHODS FOR TREATING MACULAR EDEMA AND OCULAR ANGIOGENESIS USING AN ANTI-INFLAMMATORY AGENT AND A RECEPTOR TYROSINE KINASE INHIBITOR

(76) Inventor: David P. Bingaman, Weatherford, TX (US)

Correspondence Address: Teresa J. Schultz;IP Legal (TB4-8), Alcon Research, Ltd., 6201 South Freeway, Fort Worth, TX, 76134-2099, US

(21) Appl. No.: 11/962,338

(22) Filed: Dec. 21, 2007

Related U.S. Application Data
(60) Provisional application No. 60/871,409, filed on Dec. 21, 2006.

(51) Int. Cl. A61K 31/5377; A61K 31/423; A61K 31/416

(52) U.S. Cl. 514/233.8; 514/379; 514/406

(57) ABSTRACT

The present invention provides methods for inhibiting increased vascular permeability and/or pathologic ocular angiogenesis via administration of a combination of one or more molecules that potently inhibit select receptor tyrosine kinases (RTKs) or vascular endothelial growth factor (VEGF) and one or more anti-inflammatory agents.

### RELATED APPLICATION

The present application claims priority to U.S. Provisional Patent Application No. 60/871,409 filed Dec. 21, 2006.

### BACKGROUND OF THE INVENTION

1. Field of the Invention

The present invention is directed to the prevention and treatment of macular edema and/or ocular angiogenesis, specifically diabetic macular edema, exudative age-related macular degeneration and/or proliferative diabetic retinopathy. In particular, the present invention is directed to the use of certain formulations including a COX-II inhibitor, such as a derivative of 3-benzoylphenylacetic acid, and a receptor tyrosine kinase (RTK) inhibitor to treat such disorders.

2. Description of the Related Art

Diabetes mellitus is characterized by persistent hyperglycemia that produces reversible and irreversible pathologic changes within the microvasculature of various organs. Diabetic retinopathy (DR), therefore, is a retinal microvascular disease that is manifested as a cascade of stages with increasing levels of severity and a worsening prognosis for vision. Retinal neuronal damage during diabetes mellitus may result secondary to the microangiopathy, or as some evidence suggests, it may be a direct result of hyperglycemia on retinal neurons. DR is broadly classified into 2 major clinical stages: nonproliferative diabetic retinopathy (NPDR) and proliferative diabetic retinopathy (PDR), where the term “proliferative” refers to the presence of preretinal neovascularization (PNV) emanating from the retina into the vitreous. NPDR encompasses a range of clinical subcategories which include initial “background” DR, where small multifocal changes are observed within the inner retina (e.g., microaneurysms, “dot-blot” hemorrhages, and nerve fiber layer infarcts), through preproliferative DR, which immediately precedes the development of posterior segment neovascularization (PSNV). Diabetic macular edema (DME) can be seen during either stage of DR, however, it often is observed in the latter stages of NPDR and is a prognostic indicator for progression into the most severe stage, PDR.

Although PDR is the major cause of legal blindness in patients with diabetes mellitus, macular edema is the most common cause of vision loss. Nonproliferative diabetic retinopathy (NPDR) and subsequent macular edema are associated, in part, with retinal ischemia that results from the retinal microvasculopathy induced by persistent hyperglycemia. Data accumulated from animal models and empirical human studies show that retinal ischemia is often associated with increased local levels of proinflammatory and/or proangiogenic growth factors and cytokines, such as prostaglandin E₂, vascular endothelial growth factor (VEGF), insulin-like growth factor-1 (IGF-1), etc. These molecules can alter the retinal microvasculature and cause pathologic changes such as extracellular matrix remodeling, retinal vascular leakage leading to edema, and ultimately pathologic ocular angiogenesis. The cumulative impact of these diabetic changes (altered growth factor production, hypoxia, increased sorbitol metabolism, change in redox potential, advanced glycosylation endproducts, etc.) can lead to apoptotic cell death of endothelial cells, pericytes and neuronal cells within the retina.

Diabetic macular edema and leakage from preretinal neovascular membranes is primarily associated with abnormally enhanced vascular leakage leading to interstitial edema. In addition, removal of fluid from the diabetic retina may be impaired. Fluid removal is mediated in large part by the retina pigmented epithelium (RPE), where these outer retinal cells actively pump ions and fluid away from the photoreceptors. Dysfunctional RPE pumping mechanisms may also be associated with exudative (wet) AMD. The term “exudative” refers to the increased vascular permeability of the pathologic new choroidal vessels, where the enhanced vascular permeability leads to subretinal fluid accumulation and intraretinal edema. Moreover, retinal edema can be observed in various other posterior segment diseases, such as posterior uveitis, branch or central retinal vein occlusion, surgically induced inflammation, endophthalmitis (sterile and non-sterile), scleritis, and episcleritis, etc. Regardless of disease etiology, when the edema involves the fovea, visual acuity is threatened.

Although posterior segment NV (PSNV) and macular/retinal edema are major causes of vision loss in adults, viable, approved treatment options are currently limited. The approved treatments for exudative AMD are photodynamic therapy with VISUDYNE® (QLT/Novartis) and intravitreal injection of Macugen® (pegaptanib) (Eyetech/Pfizer) or Lucentis® (ranibizumab) (Genentech). Laser photocoagulation alone or photodynamic therapy (PDT) with VISUDYNE® are therapies that involve laser-induced occlusion of the affected vasculature, which can result in localized damage to the retina. Macugen® (Eyetech/Pfizer) is an anti-VEGF aptamer that binds to VEGF₁₆₅ preventing ligand-receptor interaction and is labeled for intravitreal injections every 4 weeks. Lucentis® (Genentech) is a humanized anti-VEGF antibody fragment that also binds directly to all isoforms of human VEGF and is labeled for intravitreal injections every 6 weeks. Phase III trial results demonstrate the ability of Lucentis® to not only stabilize, but improve visual acuity in up to 35-40% of patients treated at 24 months. Late stage clinical trials are on-going in patients with diabetic macular edema using both Macugen® and Lucentis®. A variety of other pharmacologic therapies are undergoing clinical evaluation for exudative AMD, such as RETAANE® 15 mg (anecortave acetate suspension, Alcon Research, Ltd.), Envision (squalamine, Genera), the VEGF R₁R₂ Trap, (Regeneron), Cand5 (anti-VEGF siRNA, Acuity), Sirna-027 (anti-VEGFR₁ siRNA, SIRNA/Allergan), a topical receptor tyrosine kinase antagonist (TargeGen), sirolimus (rapamycin, MacuSight), etc. Several of these agents also are under investigation for use in DME.

Grid and pan retinal laser photocoagulation are the only proven options currently available for patients with diabetic macular edema or PDR, respectively. Multifocal laser photocoagulation may reduce retinal ischemia and inhibit angiogenesis by destroying healthy tissue and thus decreasing the sum metabolic demand of the retina. It also may modulate the expression and production of various cytokines and trophic factors. Unfortunately, laser photocoagulation is a cytodestructive procedure and the visual field of the treated eye is irreversibly compromised. Surgical interventions, such as vitrectomy and removal of preretinal membranes, are widely used with or without laser treatment. Similar to the exudative AMD trials, various pharmacologic agents are in clinical trials for DME, such as ARXXANT™ (ruboxystaurin mesylate, Lilly), RETISERT™ (fluocinolone acetonide, Bausch & Lomb), Posurdex (fluocinolone acetonide erodible implant, Occulex/Allergan), I-vation (nonerodible Dexamethasone implant, Occulex), Medidur (fluocinolone acetonide erodible implant, Alimera), etc. Intravitreal or periocular injection of triamcinolone acetonide, a corticosteroid (Kenalog®, Schering-Plough), and intravitreal Avastin® (anti-VEGF Mab (bevacizumab), Genentech) are also being used “off-label” for the treatment of both macular edema and wet AMD.

Subgroups of endothelial-selective Receptor Tyrosine Kinases (RTKs) are critical signaling mediators during microvascular biology and disease. RTKs comprise a large family of transmembrane receptors that mediate intracellular signaling via autophosphorylation of tyrosine residues following ligand binding. Signaling through RTKs appears to determine microvascular endothelial cell phenotype, homeostasis, and survival. Key growth factor pathways known for the retinal and choroidal vascular beds are vascular endothelial growth factor (VEGF), angiopoeitins, ephrins, fibroblast growth factor-2 (bFGF), platelet-derived growth factor, etc. For example, VEGF binds the high affinity membrane-spanning tyrosine kinase receptors VEGFR-1 (Flt-1), VEGFR-2 (KDR or Flk-1), and VEGFR-3 (Flt-4). Cell culture and gene knockout experiments indicate that VEGFR-2 and -1 are most important for endothelial physiology, vasculogenesis, and pathologic angiogenesis. More specifically, VEGFR-2 has been shown to be responsible for endothelial cell migration, proliferation, and barrier function in culture. VEGFR-2 is upregulated during retinal ischemia and pathologic ocular angiogenesis in animal models, whereas KDR blockade inhibits these abnormalities. Similarly, Angiopoietin 1 and 2 bind Tie-2, where Tie-2 signaling appears to be important in both normal vascular development and pathologic angiogenesis (ref. Hacket S F et al. J Cell Physiol 2000 184:275-284) and works in concert with VEGF signaling. Protein kinases and RTKs have been targeted for designing novel pharmacologic strategies for a variety of human conditions, such as cancer and posterior segment disease (Lawrence 1998; Gschwind 2004). Consequently, numerous pharmaceutical companies have developed medicinal chemistry efforts to design both selective and multi-targeted RTK inhibitors (Traxler 2001; Murakata 2002). Highly specific inhibitors of VEGFR-2, or KDR, have been designed and demonstrate potent and efficacious inhibition of tumor-induced angiogenesis (Shaheen 2001; Boyer 2002, Bilodeau 2002; Manley 2002; and Curtin 2004). The RTKi compound, SU11248 (Sutent®, Pfizer) was recently approved for oral use in cancer treatment. This compound was selected based on the performance of inhibitors with varying kinase selectivities in a transgenic mouse model of pancreatic islet cell carcinogenesis (Inoue 2002; McMahon 2002). In this model, the combination of a selective KDR inhibitor (SU5416) plus Gleevec, a PDGFR and KIT inhibitor, produced responses greater than either agent given individually (Bergers 2003). These responses included regressions of established tumors and were attributed to simultaneous inhibition of VEGF signaling in endothelial cells and PDGF signaling in pericytes, since a disruption of endothelial cell-pericyte association was observed. Significantly, no such disruption of endothelial cell-pericyte junctions was seen in the non-tumor vasculature from these animals.

It has been discovered that it is preferable to selectively inhibit a specific combination or combinations of Receptor Tyrosine Kinase (RTK) receptors. For example, co-pending US application no. US 2006/0189608 discusses the use of RTK inhibitors that block tyrosine autophosphorylation of VEGFR-1 (Flt-1), VEGFR-2 (KDR), VEGFR-3 (Flt-4), Tie-2, PDGFR, c-KIT, Flt-3, and CSF-1R with an IC₅₀ value of from 0.1 nM to 250 nM for each of these receptors, to inhibit ocular neovascularization and/or retinal edema.

Glucocorticoids have been used by the medical community to treat certain disorders of the back of the eye, in particular: Kenalog (triamcinolone acetonide), Celestone Soluspan (betamethasone sodium phosphate), Depo-Medrol (methylprednisolone acetate), Decadron (dexamethasone sodium phosphate), Decadron L. A. (dexamethasone acetate), and Aristocort (triamcinolone diacetate). These products are commonly administered via a periocular injection for the treatment of inflammatory disorders. Because of the lack of efficacious and safe therapies, there is a growing interest in using glucocorticoids for the treatment of, for example, retinal edema and age-related macular degeneration (AMD) via intravitreal administration. Bausch & Lomb and Control Delivery Systems are evaluating fluocinolone acetonide delivered via an intravitreal implant for the treatment of macular edema. Oculex Pharmaceuticals is studying an intravitreal dexamethasone implant for persistent macular edema. In addition, ophthalmologists are experimenting with intravitreal injection of Kenalog for the treatment of recalcitrant cystic diabetic macular edema and for exudative AMD. Although glucocorticoids are effective in treating many ocular conditions, there are significant side effects associated with the available products. Side effects include: endopthalmitis, cataracts, and elevated intraocular pressure (IOP). Although some side effects are due to the glucocorticoid itself, some may result from, or be exacerbated by, excipients in the formulations and the method of delivery.

The topical ocular use of NSAIs includes the maintenance of pupillary dilation during surgery, control of inflammation after cataract extraction and following argon laser trabeculoplasty. They are also used for non-surgically induced inflammatory disorders of the eye, such as, allergic conjunctivitis and pain following radial keratotomy or excimer laser procedures. Several topical ocular formulations are available: flurbiprofen (Ocufen®, Allergan), diclofenac (Voltaren®, Ciba Vision), and Ketorolac (Acular®, Allergan), see Ophthalmic Drug Facts, 1999, pp. 82-83 and 90-93. Topical ocular delivery of the specific NSAI in this application has been shown to decrease VEGF levels in the retina, block preretinal and choroidal NV, and prevent the development of NPDR in animal models. There is a need for NSAI formulations that are effective in treating pathologic ocular neovascularization, specifically within the posterior segment, while causing no or lessened adverse reactions. Furthermore, there are no NSAIs developed for treating persons suffering from ocular edema and/or NPDR. The formulations of this invention meet those needs.

### SUMMARY OF THE INVENTION

The present invention overcomes these and other drawbacks of the prior art by providing methods for inhibiting increased vascular permeability and/or pathologic ocular angiogenesis and providing neuroprotection of the affected retina via administration of a combination of one or more molecules that potently inhibit select receptor tyrosine kinases (RTKs) or vascular endothelial growth factor (VEGF) and one or more anti-inflammatory agents, such as COX II inhibitors, COX I/II inhibitors and glucocorticoids.

### BRIEF DESCRIPTION OF THE DRAWINGS

The following drawings form part of the present specification and are included to further demonstrate certain aspects of the present invention. The invention may be better understood by reference to these drawings in combination with the detailed description of specific embodiments presented herein.

FIG. 1 Compound 86 inhibits preretinal neovascularization (NV) following a single intravitreal injection in the rat model of oxygen-induced retinopathy (OIR).

FIG. 2 Compound 86 prevents preretinal neoavascularization (NV) following oral gavage in the rat model of oxygen-induced retinopathy (OIR).

FIG. 3 Compound 86 inhibits laser-induced choroidal neovascularization (CNV) following a single intravitreal injection in the mouse.

FIG. 4 Compound 86 induces regression of existing laser-induced choroidal neovascularization (CNV) following a single intravitreal injection in the mouse.

FIG. 5 Comparison of CNV lesions between Compound 86-treated groups in the mouse.

FIG. 6 Compound 86 inhibits laser-induced choroidal neovascularization (CNV) following oral gavage in the mouse.

FIG. 7 Compound 86 inhibits diabetes-induced retinal vascular permeability following a single intravitreal injection in the rat.

FIG. 8 Compound 86 inhibits VEGF-induced retinal vascular permeability following a single intravitreal injection in the rat.

FIG. 9 Compound 86 completely prevents diabetes-induced retinal vascular permeability following oral gavage in the STZ rat model.

FIG. 10A-FIG. 10K The Topical NSAI [nepafenac] inhibits laser-induced choroidal neovascularization in a mouse model of FIG. 10A illustrates the large areas of CNV seen in eyes treated with vehicle drops (n=20); FIG. 10B illustrates the CNV lesions in eyes treated with 0.5% nepafenac (n=27); FIG. 10C illustrates the CNV lesions in eyes treated with 0.1% nepafenac (n=28); FIG. 10D illustrates large areas of CNV seen in vehicle-treated eyes; FIG. 10E illustrates CNV lesions in eyes treated with 0.5% nepafenac; FIG. 10F illustrates CNV lesions in eyes treated with 0.1% nepafenac; FIG. 10G illustrates CNV lesions in eyes treated with 0.1% nepafenac (n=38) in a second independent experiment; FIG. 10H illustrates CNV lesions in eyes treated with 0.1% diclofenac (n=20); FIG. 10I illustrates CNV lesions in eyes treated with 0.5% ketorolac tromethamine (n=10); FIG. 10J illustrates areas of CNV at Bruch's membrane rupture sites in eyes treated with 0.5% or 0.1% nepafenac (*P<0.0001; **P=0.0005 by linear mixed model); FIG. 10K illustrates that eyes treated with 0.1% or 0.03% nepafenac, but not those treated with 0.1% diclofenac, four times a day for 2 weeks, had significantly smaller areas of CNV at Bruch's membrane rupture sites than did eyes treated with vehicle (*P=0.052; **P=0.048 by linear mixed model).

FIG. 11A-FIG. 11H The Topical NSAI [nepafenac] inhibits preretinal neovascularization in the mouse model of oxygen-induced retinopathy. Mice treated with vehicle drops (n=12) showed extensive preretinal NV (FIG. 11A, arrows in FIG. 11B). Compared with mice treated with vehicle, mice treated with 0.5% (n=13; FIG. 11C, arrows in FIG. 11D) or 0.1% (n=14; FIG. 11E; arrows in FIG. 11F) nepafenac had significantly less preretinal NV (FIG. 11G). There is marked upregulation of VEGF mRNA in hypoxic compared with those treated with vehicle (FIG. 11H).

FIG. 12 The Topical NSAI [nepafenac] inhibits morphologic vascular changes present at 9 months of diabetes in the STZ-diabetic rat.

### DETAILED DESCRIPTION PREFERRED EMBODIMENTS

According to the methods of the present invention, a composition comprising an anti-inflammatory agent, such as a derivative of 3-benzoylphenylacetic acid, and a composition comprising a RTK inhibitor are administered to a patient suffering from diabetic macular edema and/or ocular angiogenesis in order to prevent the loss of visual acuity associated with such conditions. The anti-inflammatory agent and the RTK inhibitor may be administered together in the same composition, or they may be administered separately, in different compositions.

It is important that the RTKi compounds for use in the methods of the invention exhibit a receptor binding profile where multiple receptors in the RTK family are blocked by a single compound. One preferred group of receptors for which tyrosine autophosphorylation is blocked includes VEGF receptor 1 (Flt-1), VEGF receptor 2 (KDR), VEGF receptor 3 (Flt-4), Tie-2, PDGFR, c-KIT, Flt-3, and CSF-1R. Additional preferred binding profiles include the following: a) Tie-2, PDGFR, and VEGF receptor 2 (KDR); b) VEGF receptor 2 (KDR), VEGF receptor 1 (Flt-1), PDGFR, and Tie-2; c). VEGF receptor 2 (KDR), VEGF receptor 1 (Flt-1), and Tie-2; d) VEGF receptor 2 (KDR), VEGF receptor 1 (Flt-1), and PDGFR; e) VEGF receptor 2 (KDR) and Tie-2; f) VEGF receptor 2 (KDR) and PDGFR; and g) VEGF receptor 2 (KDR), Tie-2, and PDGFR.

Preferred RTK inhibitor compounds for use in the methods of the present invention are potent, competitive inhibitors of the ATP binding site for a select group of RTKs. That is, preferred agents simultaneously block tyrosine autophosphorylation of VEGFR-1 (Flt-1), VEGFR-2 (KDR), VEGFR-3 (Flt-4), TIE-2, PDGFR, c-KIT, FLT-3, and CSF-1R, or some combination of two or more of these receptors, at low nM concentrations. Preferably, RTK inhibitor compounds for use in the methods of the invention exhibit an IC₅₀ range between 0.1 nM and 250 nM for each of these receptors. More preferred RTK inhibitor compounds exhibit an IC₅₀ range between 0.1 nM and 100 nM for at least six of these receptors. Most preferred RTK inhibitor compounds possess an IC₅₀ range between 0.1 nM and 10 nM for at least four of these receptors.

In one preferred aspect, for each grouping of receptors listed in a)-g) above, the IC₅₀ value of each receptor in each group will be from 0.1 nM to 200 nM. In another preferred aspect, the IC₅₀ value of each receptor in each group will be from 0.1 nM to 100 nM. In yet another preferred embodiment, at least one receptor in each preferred group of receptors listed in a)-g) above will exhibit an IC₅₀ value of less than 10 nM. In yet another preferred embodiment, two or more receptors in each preferred group of receptors listed in a)-g) above will exhibit an IC₅₀ value of less than 10 nM.

Preferred RTKi compounds for use in the compositions and methods of the invention include, but are not limited to, the compounds listed in Table 1:

TABLE 1

| No. | Compound Name |
|---|---|
| 1 | N-[4-(3-amino-1,2-benzisoxazol-4-yl)phenyl]-N′-(3-methylphenyl)urea |
| 2 | N-[4-(3-amino-1,2-benzisoxazol-4-yl)phenyl]-N′-[2-(trifluoromethyl)phenyl]urea |
| 3 | N-[4-(3-amino-1,2-benzisoxazol-4-yl)phenyl]-N′-(2-fluoro-5-methylphenyl)urea |
| 4 | N-[4-(3-amino-1,2-benzisoxazol-4-yl)phenyl]-N′-[3-(trifluoromethyl)phenyl]urea |
| 5 | N-[4-(3-amino-1,2-benzisoxazol-4-yl)phenyl]-N′-[2-fluoro-5- |
| | (trifluoromethyl)phenyl]urea |
| 6 | N-[4-(3-amino-7-methoxy-1,2-benzisoxazol-4-yl)phenyl]-N′-[2-fluoro-5- |
| | (trifluoromethyl)phenyl]urea |
| 7 | N-[4-(3-amino-7-methoxy-1,2-benzisoxazol-4-yl)phenyl]-N′-(3-methylphenyl)urea |
| 8 | N-[4-(3-amino-7-methoxy-1,2-benzisoxazol-4-yl)phenyl]-N′-[3- |
| | (trifluoromethyl)phenyl]urea |
| 9 | N-[4-(3-amino-7-methoxy-1,2-benzisoxazol-4-yl)phenyl]-N′-(3-chlorophenyl)urea |
| 10 | N-[4-(3-amino-7-methoxy-1,2-benzisoxazol-4-yl)phenyl]-N′-(2-fluoro-5- |
| | methylphenyl)urea |
| 11 | N-{4-[3-amino-7-(4-morpholinylmethyl)-1,2-benzisoxazol-4-yl]phenyl}-N′-[2-fluoro- |
| | 5-(trifluoromethyl)phenyl]urea |
| 12 | N-{4-[3-amino-7-(4-morpholinylmethyl)-1,2-benzisoxazol-4-yl]phenyl}-N′-[3- |
| | (trifluoromethyl)phenyl]urea |
| 13 | N-{4-[3-amino-7-(4-morpholinylmethyl)-1,2-benzisoxazol-4-yl]phenyl}-N′-(3- |
| | chlorophenyl)urea |
| 14 | N-{4-[3-amino-7-(4-morpholinylmethyl)-1,2-benzisoxazol-4-yl]phenyl}-N′-(3- |
| | methylphenyl)urea |
| 15 | N-{4-[3-amino-7-(4-morpholinylmethyl)-1,2-benzisoxazol-4-yl]phenyl}-N′-(2-fluoro- |
| | 5-methylphenyl)urea |
| 16 | N-{4-[3-amino-7-(4-morpholinylmethyl)-1,2-benzisoxazol-4-yl]phenyl}-N′-(3,5- |
| | dimethylphenyl)urea |
| 17 | N-{4-[3-amino-7-(4-morpholinylmethyl)-1,2-benzisoxazol-4-yl]phenyl}-N′-(3- |
| | phenoxyphenyl)urea |
| 18 | N-{4-[3-amino-7-(4-morpholinylmethyl)-1,2-benzisoxazol-4-yl]phenyl}-N′-(3- |
| | bromophenyl)urea |
| 19 | N-(4-{3-amino-7-[2-(4-morpholinyl)ethoxy]-1,2-benzisoxazol-4-yl}phenyl)-N′-[3- |
| | (trifluoromethyl)phenyl]urea |
| 20 | N-(4-{3-amino-7-[2-(4-morpholinyl)ethoxy]-1,2-benzisoxazol-4-yl}phenyl)-N′-(2- |
| | fluoro-5-methylphenyl)urea |
| 21 | N-(4-{3-amino-7-[2-(4-morpholinyl)ethoxy]-1,2-benzisoxazol-4-yl}phenyl)-N′-[2- |
| | fluoro-5-(trifluoromethyl)phenyl]urea |
| 22 | N-(4-{3-amino-7-[2-(4-morpholinyl)ethoxy]-1,2-benzisoxazol-4-yl}phenyl)-N′-(3- |
| | methylphenyl)urea |
| 23 | N-[4-(3-amino-1,2-benzisoxazol-4-yl)phenyl]-N′-(3,5-dimethylphenyl)urea |
| 24 | N-[4-(3-amino-1,2-benzisoxazol-4-yl)phenyl]-N′-phenylurea |
| 25 | N-[4-(3-amino-1,2-benzisoxazol-4-yl)phenyl]-N′-(4-methylphenyl)urea |
| 26 | N-[4-(3-amino-1,2-benzisoxazol-4-yl)phenyl]-N′-(3-cyanophenyl)urea |
| 27 | N-[4-(3-amino-1,2-benzisoxazol-4-yl)phenyl]-N′-[4-fluoro-3- |
| | (trifluoromethyl)phenyl]urea |
| 28 | N-[4-(3-amino-1,2-benzisoxazol-4-yl)phenyl]-N′-(3-bromophenyl)urea |
| 29 | N-[4-(3-amino-1,2-benzisoxazol-4-yl)phenyl]-N′-(3-chlorophenyl)urea |
| 30 | N-[4-(3-amino-1,2-benzisoxazol-4-yl)phenyl]-N′-(3-ethylphenyl)urea |
| 31 | N-[4-(3-amino-1,2-benzisoxazol-4-yl)phenyl]-N′-[4-(trifluoromethyl)phenyl]urea |
| 32 | N-[4-(3-amino-1,2-benzisoxazol-4-yl)phenyl]-N′-(3-fluoro-4-methylphenyl)urea |
| 33 | N-[4-(3-amino-1,2-benzisoxazol-4-yl)phenyl]-N′-(3-fluorophenyl)urea |
| 34 | N-[4-(3-amino-1,2-benzisoxazol-4-yl)phenyl]-N′-(3,5-difluorophenyl)urea |
| 35 | N-[4-(3-amino-1,2-benzisoxazol-4-yl)phenyl]-N′-(3-methoxyphenyl)urea |
| 36 | N-[4-(3-amino-1,2-benzisoxazol-4-yl)phenyl]-N′-(4-methoxyphenyl)urea |
| 37 | N-[4-(3-amino-1,2-benzisoxazol-4-yl)phenyl]urea |
| 38 | N-[4-(3-amino-1,2-benzisoxazol-4-yl)phenyl]-N′-(3-nitrophenyl)urea |
| 39 | N-[4-(3-amino-1,2-benzisoxazol-4-yl)phenyl]-N′-(4-fluorophenyl)urea |
| 40 | N-[4-(3-amino-1,2-benzisoxazol-4-yl)phenyl]-N′-(2-fluorophenyl)urea |
| 41 | N-[4-(3-amino-1,2-benzisoxazol-4-yl)phenyl]-N′-(3-chloro-4-fluorophenyl)urea |
| 42 | N-[4-(3-amino-1,2-benzisoxazol-4-yl)phenyl]-N′-(3-chloro-4-methoxyphenyl)urea |
| 43 | N-[4-(3-amino-1,2-benzisoxazol-4-yl)phenyl]-N′-[4-(dimethylamino)phenyl]urea |
| 44 | N-[4-(3-amino-1,2-benzisoxazol-4-yl)phenyl]-N′-[4-(trifluoromethoxy)phenyl]urea |
| 45 | N-[4-(3-amino-1,2-benzisoxazol-4-yl)phenyl]-N′-[2-(trifluoromethoxy)phenyl]urea |
| 46 | N-[4-(3-amino-1,2-benzisoxazol-4-yl)phenyl]-N′-[3,5-bis(trifluoromethyl)phenyl]urea |
| 47 | N-[4-(3-amino-1,2-benzisoxazol-4-yl)phenyl]-N′-(3-chloro-4-methylphenyl)urea |
| 48 | N-[4-(3-amino-7-methoxy-1,2-benzisoxazol-4-yl)phenyl]-N′-[3,5- |
| | bis(trifluoromethyl)phenyl]urea |
| 49 | N-[4-(3-amino-7-methoxy-1,2-benzisoxazol-4-yl)phenyl]-N′-[4- |
| | (trifluoromethoxy)phenyl]urea |
| 50 | N-[4-(3-amino-7-methoxy-1,2-benzisoxazol-4-yl)phenyl]-N′-(3-fluorophenyl)urea |
| 51 | N-[4-(3-amino-7-methoxy-1,2-benzisoxazol-4-yl)phenyl]-N′-(3-methoxyphenyl)urea |
| 52 | N-[4-(3-amino-7-methoxy-1,2-benzisoxazol-4-yl)phenyl]-N′-(3,5-difluorophenyl)urea |
| 53 | N-[4-(3-amino-7-methoxy-1,2-benzisoxazol-4-yl)phenyl]-N′-(4-methylphenyl)urea |
| 54 | N-[4-(3-amino-7-methoxy-1,2-benzisoxazol-4-yl)phenyl]-N′-(3-bromophenyl)urea |
| 55 | N-[4-(3-amino-7-methoxy-1,2-benzisoxazol-4-yl)phenyl]-N′-(3,5-dimethylphenyl)urea |
| 56 | N-[4-(3-amino-7-methoxy-1,2-benzisoxazol-4-yl)phenyl]-N′-[4- |
| | (dimethylamino)phenyl]urea |
| 57 | N-[4-(3-amino-7-methyl-1,2-benzisoxazol-4-yl)phenyl]-N′-(3-methylphenyl)urea |
| 58 | N-[4-(3-amino-7-methyl-1,2-benzisoxazol-4-yl)phenyl]-N′-(3-chlorophenyl)urea |
| 59 | N-[4-(3-amino-7-methyl-1,2-benzisoxazol-4-yl)phenyl]-N′-(2-fluoro-5- |
| | methylphenyl)urea |
| 60 | N-[4-(3-amino-7-methyl-1,2-benzisoxazol-4-yl)phenyl]-N′-[2-fluoro-5- |
| | (trifluoromethyl)phenyl]urea |
| 61 | N-[4-(3-amino-7-methyl-1,2-benzisoxazol-4-yl)phenyl]-N′-[3- |
| | (trifluoromethyl)phenyl]urea |
| 62 | N-[4-(3-amino-7-methyl-1,2-benzisoxazol-4-yl)phenyl]-N′-(3,5-dimethylphenyl)urea |
| 63 | N-[4-(3-amino-7-methyl-1,2-benzisoxazol-4-yl)phenyl]-N′-(3-ethylphenyl)urea |
| 64 | N-[4-(3-amino-7-methyl-1,2-benzisoxazol-4-yl)phenyl]-N′-(4-methylphenyl)urea |
| 65 | N-[4-(3-amino-7-methyl-1,2-benzisoxazol-4-yl)phenyl]-N′-[4- |
| | (trifluoromethoxy)phenyl]urea |
| 66 | N-[4-(3-amino-7-methyl-1,2-benzisoxazol-4-yl)phenyl]-N′-(3-fluoro-4- |
| | methylphenyl)urea |
| 67 | N-[4-(3-amino-7-methyl-1,2-benzisoxazol-4-yl)phenyl]-N′-(3-methoxyphenyl)urea |
| 68 | N-[4-(3-amino-7-methyl-1,2-benzisoxazol-4-yl)phenyl]-N′-phenylurea |
| 69 | N-[4-(3-amino-7-methyl-1,2-benzisoxazol-4-yl)phenyl]-N′-[3,5- |
| | bis(trifluoromethyl)phenyl]urea |
| 70 | N-[4-(3-amino-7-methyl-1,2-benzisoxazol-4-yl)phenyl]-N′-(3-bromophenyl)urea |
| 71 | N-[4-(3-amino-7-methyl-1,2-benzisoxazol-4-yl)phenyl]-N′-(3-fluorophenyl)urea |
| 72 | N-[4-(3-amino-7-methoxy-1,2-benzisoxazol-4-yl)phenyl]-N′-[4-fluoro-3- |
| | (trifluoromethyl)phenyl]urea |
| 73 | N-[4-(3-amino-7-methoxy-1,2-benzisoxazol-4-yl)phenyl]-N′-(4-fluoro-3- |
| | methylphenyl)urea |
| 74 | N-[4-(3-amino-7-fluoro-1,2-benzisoxazol-4-yl)phenyl]-N′-[3- |
| | (trifluoromethyl)phenyl]urea |
| 75 | N-[4-(3-amino-7-fluoro-1,2-benzisoxazol-4-yl)phenyl]-N′-(3-chlorophenyl)urea |
| 76 | N-[4-(3-amino-7-fluoro-1,2-benzisoxazol-4-yl)phenyl]-N′-[4-fluoro-3- |
| | (trifluoromethyl)phenyl]urea |
| 77 | N-[4-(3-amino-7-fluoro-1,2-benzisoxazol-4-yl)phenyl]-N′-(3-methylphenyl)urea |
| 78 | N-[4-(3-amino-7-fluoro-1,2-benzisoxazol-4-yl)phenyl]-N′-[2-fluoro-5- |
| | (trifluoromethyl)phenyl]urea |
| 79 | N-[4-(3-amino-7-fluoro-1,2-benzisoxazol-4-yl)phenyl]-N′-(2-fluoro-5- |
| | methylphenyl)urea |
| 80 | N-{4-[3-amino-7-(trifluoromethoxy)-1,2-benzisoxazol-4-yl]phenyl}-N′-[2-fluoro-5- |
| | (trifluoromethyl)phenyl]urea |
| 81 | N-{4-[3-amino-7-(trifluoromethoxy)-1,2-benzisoxazol-4-yl]phenyl}-N′-[3- |
| | (trifluoromethyl)phenyl]urea |
| 82 | N-{4-[3-amino-7-(trifluoromethoxy)-1,2-benzisoxazol-4-yl]phenyl}-N′-(2-fluoro-5- |
| | methylphenyl)urea |
| 83 | N-{4-[3-amino-7-(trifluoromethoxy)-1,2-benzisoxazol-4-yl]phenyl}-N′-(3- |
| | chlorophenyl)urea |
| 84 | N-{4-[3-amino-7-(trifluoromethoxy)-1,2-benzisoxazol-4-yl]phenyl}-N′-(3- |
| | bromophenyl)urea |
| 85 | N-{4-[3-amino-7-(trifluoromethoxy)-1,2-benzisoxazol-4-yl]phenyl}-N′-[4-fluoro-3- |
| | (trifluoromethyl)phenyl]urea |
| 86 | N-[4-[3-amino-1H-indazol-4-yl]phenyl]-N′-(2-fluoro-5-methylphenyl)urea |
| 87 | N-{4-[3-amino-7-(trifluoromethoxy)-1,2-benzisoxazol-4-yl]phenyl}-N′-(4-fluoro-3- |
| | methylphenyl)urea |
| 88 | N-[4-(3-amino-1H-indazol-4-yl)phenyl]-N′-(3-methylphenyl)urea |
| 89 | N-[4-(3-amino-1H-indazol-4-yl)phenyl]-N′-(3,5-dimethoxyphenyl)urea |
| 90 | N-[4-(3-amino-1H-indazol-4-yl)phenyl]-N′-(3-chlorophenyl)urea |
| 91 | N-[4-(3-amino-1H-indazol-4-yl)phenyl]-N′-[3-(trifluoromethyl)phenyl]urea |
| 92 | N-[4-(3-amino-1H-indazol-4-yl)phenyl]-N′-[2-fluoro-5-(trifluormethyl)phenyl]urea |
| 93 | N-[4-(3-amino-1H-indazol-4-yl)phenyl]-N′-(3-bromophenyl)urea |
| 94 | N-[4-(3-amino-1H-indazol-4-yl)phenyl]-N′-(3-bromo-4-methylphenyl)urea |
| 95 | N-[4-(3-amino-1H-indazol-4-yl)phenyl]-N′-(3-ethylphenyl)urea |
| 96 | N-[4-(3-amino-1H-indazol-4-yl)phenyl]-N′-phenylurea |
| 97 | N-[4-(3-amino-1H-indazol-4-yl)phenyl]-N′-(3-fluoro-4-methylphenyl)urea |
| 98 | N-[4-(3-amino-1H-indazol-4-yl)phenyl]-N′-(2-fluorophenyl)urea |
| 99 | N-[4-(3-amino-1H-indazol-4-yl)phenyl]-N′-(4-fluorophenyl)urea |
| 100 | N-[4-(3-amino-1H-indazol-4-yl)phenyl]-N′-(3-fluorophenyl)urea |
| 101 | N-[4-(3-amino-1H-indazol-4-yl)phenyl]-N′-(3-hydroxyphenyl)urea |
| 102 | N-[4-(3-amino-1H-indazol-4-yl)phenyl]-N′-(3-methylphenyl)urea |
| 103 | N-[4-(3-amino-1H-indazol-4-yl)-2-fluorophenyl]-N′-(2-fluoro-5-methylphenyl)urea |
| 104 | N-[4-(3-amino-1H-indazol-4-yl)phenyl]-N′-[4-fluoro-3-(trifluoromethyl)phenyl]urea |
| 105 | N-[4-(3-amino-1H-indazol-4-yl)phenyl]-N′-[2-fluoro-3-(trifluoromethyl)phenyl]urea |
| 106 | N-[4-(3-amino-1H-indazol-4-yl)phenyl]-N′-(4-bromo-2-fluorophenyl)urea |
| 107 | N-[4-(3-amino-1H-indazol-4-yl)phenyl]-N′-(5-fluoro-2-methylphenyl)urea |
| 108 | N-[4-(3-amino-1H-indazol-4-yl)phenyl]-N′-(4-fluoro-3-methylphenyl)urea |
| 109 | N-[4-(3-amino-1H-indazol-4-yl)phenyl]-N′-[2-fluoro-5-(hydroxymethyl)phenyl]urea |
| 110 | 3-[({[4-(3-amino-1H-indazol-4-yl)phenyl]amino}carbonyl)amino]-4-fluorobenzoic |
| | acid |
| 111 | Methyl 3-[({[4-(3-amino-1H-indazol-4-yl)phenyl]amino}carbonyl)amino]-4- |
| | fluorobenzoate |

Preferred RTKi compounds for use in the methods of the invention include Compounds 86 and 88-111. The most preferred RTKi compound for use in the methods of the invention is Compound 86.

Vascular growth in the retina leads to visual degeneration culminating in blindness. Vascular endothelial growth factor (VEGF) accounts for most of the angiogenic activity produced in or near the retina in diabetic retinopathy. Ocular VEGF mRNA and protein are elevated by conditions such as retinal vein occlusion in primates and decreased pO₂ levels in mice that lead to neovascularization. Intraocular injections of either anti-VEGF monoclonal antibodies or VEGF receptor immunofusions inhibit ocular neovascularization in rodent and primate models. Regardless of the cause of induction of VEGF in human diabetic retinopathy, inhibition of ocular VEGF is useful in treating the disease.

Thus, it is further contemplated that compounds targeting VEGF receptors would be useful in combination with the anti-inflammatory compounds disclosed herein for use in treating diabetic macular edema and/or ocular angiogenesis in order to prevent the loss of visual acuity associated with such conditions and to provide additive or synergistic activity within the retinal tissues. Acceptable anti-VEGF compounds for use in the methods of the invention include any molecule that binds directly to VEGF and prevents ligand-receptor interaction (i.e., Macugen® (pegaptanib), Lucentis® (ranibizumab), Avastin® (bevacizumab), VEGF Trap, or any agent known to down-regulate VEGF production (i.e., siRNA molecules Cand5, Sirna-027), directly or indirectly. Other known anti-angiogenic agents, such as anecortave acetate, anecortave desacetate, corticosteroids, HIF-1 inhibitors (e.g., rapamycin and its analogs), etc., have been shown to down regulate VEGF and, may also be used in the compositions and methods of the invention.

The models described in the examples below can be used to identify additional effective RTK inhibitor or anti-VEGF compounds for potential use in the methods of the invention, or to select preferred compounds from those identified via receptor binding assays that are well known to the skilled artisan. For example, a test compound may be evaluated in the rat OIR model described in Example 1, the mouse laser model described in Example 3, the rat VEGF model described in Example 6, and the diabetic rat model described in Example 7. Potential RTK inhibitors or anti-VEGF compounds (test compounds) for use in the methods of the invention, preferably provide:
>75% inhibition of preretinal NV in the rat OIR model following a single intravitreal injection of ≦3% solution or suspension, or oral gavage with a solution or suspension ≦30 mg/kg/d.
>70% inhibition of choroidal NV in the mouse laser model following a single intravitreal injection of ≦3% solution or suspension, or oral gavage with a solution or suspension <30 mg/kg/d.
>50% inhibition of retinal vascular permeability in rat VEGF model following a single intravitreal injection of ≦3% solution or suspension, or oral gavage with a solution or suspension <30 mg/kg/d.
>75% inhibition of retinal vascular permeability in the STZ-induced diabetic rat model following a single intravitreal injection of ≦3% solution or suspension, or oral gavage with a solution or suspension <30 mg/kg/d.

Compounds that are able to achieve activity in the categories described above are preferred agents with potential clinical utility. More preferred agents exhibit >25% regression of choroidal NV in the mouse laser model following a single intravitreal injection of ≦3% solution or suspension, or 50% regression with oral gavage with a solution or suspension <30 mg/kg/d.

According to the present invention, a therapeutically effective amount of an anti-inflammatory compound is administered topically, locally or systemically, in combination with a RTK inhibiting or anti-VEGF compound, to treat or prevent diabetic macular edema and/or ocular angiogenesis. It is contemplated that virtually any agent capable of preventing or treating inflammation will be useful in the compositions and methods of the present invention. Preferred anti-inflammatory agents include COXII or COXI/II inhibitors, such as nepafenac, amfenac, diclofenac, ketorelac, glucocorticoids, etc.

Preferred COXII inhibitors for use in the compositions and methods of the present invention include derivatives of 3-benzoylphenylacetic acid. It is known that some derivatives of 3-benzoylphenylacetic acid, such as nepafenac, may be administered topically to treat conditions of the back of the eye because nepafenac will penetrate the anterior segment tissues and achieve bioactive concentration in the retina and choroid. Other derivatives of 3-benzoylphenylacetic acid, such as amfenac, will not penetrate to the back of the eye and must be delivered directly to the site of desired treatment. Direct intravitreal administration of either or both agents is feasible and the proof-of-concept has been demonstrated in preclinical models. Intravitreal platforms include aqueous and nonaqueous solutions, aqueous and nonaqueous suspensions, bioerodible implants, and nonerodible implants.

3-benzoylphenylacetic acid and certain of its derivatives are known to possess anti-inflammatory activity. U.S. Pat. Nos. 4,254,146, 4,045,576, 4,126,635, and 4,503,073, and U.K. Patent Application Nos. 2,071,086A and 2,093,027A disclose various 3-benzoylphenylacetic acids, salts and esters, and hydrates thereof, having anti-inflammatory activity. U.S. Pat. No. 4,568,695 discloses 2-amino-3-benzoylphenylethyl alcohols having anti-inflammatory activity. U.S. Pat. No. 4,313,949 discloses 2-amino-3-benzoyl-phenylacetamides having anti-inflammatory activity.

Certain derivatives of 2-amino-3-benzoylbenzeneacetic acid (amfenac) and 2-amino-3-(4-chloro-benzoyl)benzeneacetic acid have also been evaluated by Walsh et al. (1990), in an attempt to discover nonsteroidal anti-inflammatory prodrugs with minimal or no gastrointestinal side effects upon oral administration.

U.S. Pat. No. 4,683,242 describes the transdermal administration of 2-amino-3-benzoylphenylacetic acids, salts, and esters, and hydrates and alcohols thereof to control inflammation and alleviate pain.

U.S. Pat. No. 4,910,225 describes certain benzoylphenylacetic acids for local administration to control ophthalmic, nasal or otic inflammation. Only acetic acids are disclosed in the '225 patent; no esters or amides are mentioned or taught as anti-inflammatory agents for local administration to the eyes, nose and ears.

U.S. Pat. No. 5,475,034 describes topically administrable compositions containing certain amide and ester derivatives of 3-benzyolphenylacetic acid, including nepafenac, useful for treating ophthalmic inflammatory disorders and ocular pain. According to the '034 patent, “[s]uch disorders include, but are not limited to uveitis, scleritis, episcleritis, keratitis, surgically-induced inflammation, and endophthalmitis.” The '034 patent contains no discussion of the use of nepafenac alone, or in combination with a RTK inhibitor, for the treatment of diabetic retinal edema and/or associated ocular angiogenesis.

U.S. Pat. No. 6,066,671 describes the topical use of certain amide and ester derivatives of 3-benzoylphenylacetic acid, including nepafenac, for treating GLC1A glaucoma. Its use in combination with a RTK inhibiting compound has not been suggested as a means for treating diabetic macular edema and/or ocular angiogenesis.

The 3-benzoylphenylacetic acids and derivatives useful in the methods of the present invention are those of formula (I) below.

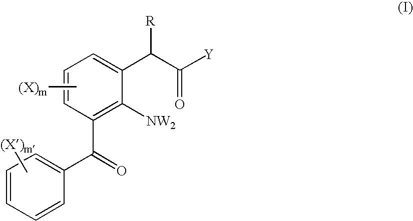

wherein

R═H, C₁₋₄ (un)branched alkyl, CF₃, SR⁴;

Y═OR′, NR″R′;

R′=H, C₁₋₁₀ (un)branched alkyl, (un)substituted aryl (substitution as defined by X below), (un)substituted heterocycle (substitution as defined by X below),

—(CH₂)ₙZ(CH₂)_{n′}A;

n=2-6;

n′=1-6;

Z=nothing, O, C═O, OC(═O), C(═O)O, C(═O)NR³, NR³C(═O), S(O)ₙ_{²}, CHOR³, NR³;

n²=0-2;

R³=H, C₁₋₆ (un)branched alkyl, (un)substituted aryl (substitution as defined by X below), (un)substituted heterocycle (substitution as defined by X below);

A=H, OH, optionally (un)substituted aryl (substitution as defined by X below), (un)substituted heterocycle (substitution as defined by X below), —(CH₂)ₙOR³;

R″=H, OH, OR′;

X and X′ independently=H, F, Cl, Br, I, OR′, CN, OH, S(O)ₙ_{²}R⁴, CF₃, R⁴, NO₂;

R⁴=C₁₋₆ (un)branched alkyl;

m=0-3;

m′=0-5;

W=O, H.

As used herein, the acid (Y═OH) includes pharmaceutically acceptable salts as well.

Preferred compounds for use in the methods of the present invention are those of Formula I wherein:

R=H, C₁₋₂ alkyl;

Y=NR′R″;

R′=H, C₁₋₆ (un)branched alkyl, —(CH₂)ₙZ(CH₂)_{n′}A;

Z=nothing, O, CHOR³, NR³;

R₃=H;

A=H, OH, (un)substituted aryl (substitution as defined by X below);

X and X′ independently=H, F, Cl, Br, CN, CF₃, OR′, SR⁴, R⁴;

R″=H;

R⁴=C₁₋₄ (un)branched alkyl;

m=0-2;

m′=0-2;

W═H;

n=2-4;

n′=0-3.

The most preferred 3-benzoylphenylacetic acid derivatives for use in the compositions or method of the present invention are 2-Amino-3-(4-fluorobenzoyl)-phenylacetamide; 2-Amino-3-benzoyl-phenylacetamide (nepafenac); and 2-Amino-3-(4-chlorobenzoyl)-phenylacetamide.

Glucocorticoids have been used by the medical community to treat certain disorders of the back of the eye, in particular: Kenalog (triamcinolone acetonide), Celestone Soluspan (betamethasone sodium phosphate), Depo-Medrol (methylprednisolone acetate), Decadron (dexamethasone sodium phosphate), Decadron L. A. (dexamethasone acetate), and Aristocort (triamcinolone diacetate). These products are commonly administered via a periocular injection for the treatment of inflammatory disorders. Because of the lack of efficacious and safe therapies, there is a growing interest in using glucocorticoids for the treatment of, for example, retinal edema and age-related macular degeneration (AMD). Bausch & Lomb and Control Delivery Systems are evaluating fluocinolone acetonide delivered via an intravitreal implant for the treatment of macular edema. Oculex Pharmaceuticals is studying a dexamethasone implant for persistent macular edema. In addition, ophthalmologists are experimenting with intravitreal injection of Kenalog for the treatment of recalcitrant cystic diabetic macular edema and for exudative AMD.

Although glucocorticoids are very effective in treating many ocular conditions, there are significant side effects associated with the available products. Side effects include: endopthalmitis, cataracts, and elevated intraocular pressure (IOP). Although some side effects are due to the glucocorticoid itself, some may result from, or be exacerbated by, excipients in the formulations.

Glucocorticoids which may be employed in the present invention include all acceptable compounds which are effective in the treatment of macular edema and/or NPDR. The preferred glucocorticoids include, dexamethasone, fluoromethalone, medrysone, betamethasone, triamcinolone, triamcinolone acetonide, prednisone, prednisolone, hydrocortisone, rimexolone, and pharmaceutically acceptable salts thereof. Further examples of glucocorticoids include prednicarbate, deflazacort, halomethasone, tixocortol, prednylidene (21-diethylaminoacetate), prednival, paramethasone, methylprednisolone, meprednisone, mazipredone, isoflupredone, halopredone acetate, halcinonide, formocortal, flurandrenolide, fluprednisolone, fluprednidine acetate, fluperolone acetate, fluocortolone, fluocortin butyl, fluocinonide, fluocinolone acetonide, flunisolide, flumethasone, fludrocortisone, fluclorinide, enoxolone, difluprednate, diflucortolone, diflorasone diacetate, desoximetasone (desoxymethasone), desonide, descinolone, cortivazol, corticosterone, cortisone, cloprednol, clocortolone, clobetasone, clobetasol, chloroprednisone, cafestol, budesonide, beclomethasone, amcinonide, allopregnane acetonide, alclometasone, 21-acetoxypregnenolone, tralonide, diflorasone acetate, deacylcortivazol, RU-26988, budesonide, and deacylcortivazol oxetanone. All of the above-cited glucocorticoids are known compounds. Further information about the compounds may be found for example, in The Merck Index, Eleventh Edition (1989), and the publications cited therein, the entire contents of which are hereby incorporated in the present specification by reference.

Preferred steroids for treating chronic retinal edema and/or NPDR are less potent than many of the marketed products. For example, prednisolone, prednisolone acetate, rimexolone, fluoromethalone, and fluoromethalone acetate would be useful in such a scenario, but with reduced incidence of cataracts and/or elevated IOP.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Prevention of Preretinal Neovascularization Following Intravitreal Delivery of the Receptor Kinase Tyrosine Inhibitor (RTKi), Compound 86, in the Rat Model of Oxygen-Induced Retinopathy

METHODS: Pregnant Sprague-Dawley rats were received at 14 days gestation and subsequently gave birth on Day 22±1 of gestation. Immediately following parturition, pups were pooled and randomized into separate litters (n=17 pups/litter), placed into separate shoebox cages inside oxygen delivery chamber, and subjected to an oxygen-exposure profile from Day 0-14 postpartum. Litters were then placed into room air from Day 14/0 through Day 14/6 (days 14-20 postpartum). Additionally on Day 14/0, each pup was randomly assigned as an oxygen-exposed control or into various treatment groups. For those randomized into an injection treatment group: one eye received a 5 μl intravitreal injection of 0.1%, 0.3%, 0.6%, or 1% RTKi and the contralateral eye received a 5 μl intravitreal injection of vehicle. At Day 14/6 (20 days postpartum), all animals in both studies were euthanized.

Immediately following euthanasia, retinas from all rat pups were harvested, fixed in 10% neutral buffered formalin for 24 hours, subjected to ADPase staining, and fixed onto slides as whole mounts. Digital images were acquired from each retinal flat mount that was adequately prepared. Computerized image analysis was used to obtain a NV clockhour score from each readable sample. Each clockhour out of 12 total per retina was assessed for the presence or absence of preretinal NV. Statistical comparisons using median scores for NV clockhours from each treatment group were utilized in nonparametric analyses. Each noninjected pup represented one NV score by taking the average value of both eyes and was used in comparisons against each dosage group. Because the pups were randomly assigned and no difference was observed between oxygen-exposed control pups from all litters, the NV scores were combined for all treatment groups. P≦0.05 was considered statistically significant.

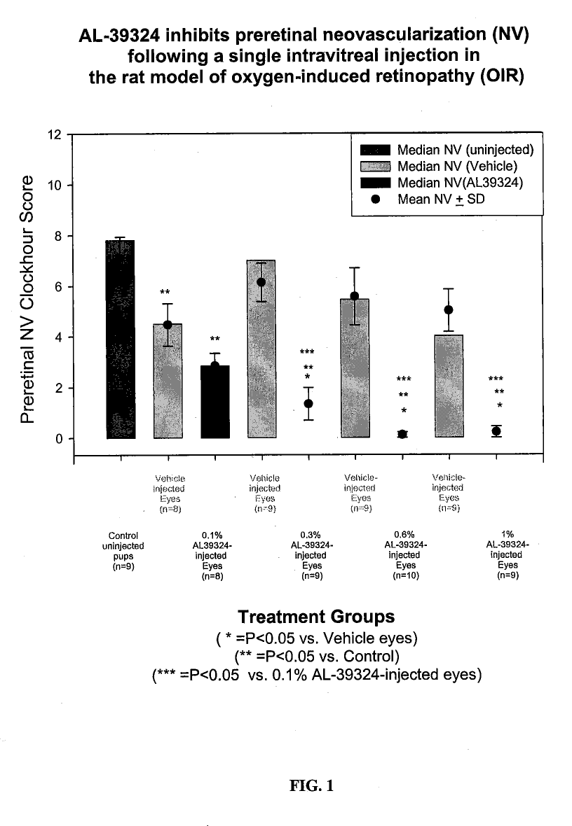

RESULTS: Local administration of RTKi provided potent anti-angiogenic efficacy against preretinal neovascularization, where 100% inhibition of preretinal NV was observed between 0.3%-1% suspensions. An overall statistical difference was demonstrated between treatment groups (Kiruskal-Wallis one-way ANOVA test: P<0.001) (FIG. 1). Eyes treated with 0.3-1% RTKi exhibited significant inhibition of preretinal NV as compared to vehicle-injected injected and control, noninjected eyes (Table 2). Efficacy was not observed in 0.1% treated eyes.

TABLE 2

| | % Inhibition | | | | Median | |
|---|---|---|---|---|---|---|
| | (vs. vehicle- | | Median | NV | NV | NV Range |
| Treatment | injected eye) | P value | NV | Range | (Vehicle) | (Vehicle) |
| Untreated control | | | 7.8 | 3.2-10.9 | | |
| 0.1% RTKi | 37 | 0.161 | 2.835 | 1.1-5.3 | 4.5 | 1.1-8.4 |
| 0.3% RTKi | 100 | 0.002 | 0 | 0-5 | 7 | 2-8.72 |
| 0.6% RTKi | 100 | <0.001 | 0 | 0-1.1 | 5.45 | 2-10.9 |
| 1% RTKi | 100 | <0.001 | 0 | 0-2 | 4 | 1-8 |

### Example 2

### Systemic Administration of RTKi (Compound 86) Potently Prevents Preretinal Neovascularization in the Rat OIR Model

METHODS: Pregnant Sprague-Dawley rats were received at 14 days gestation and subsequently gave birth on Day 22±1 of gestation. Immediately following parturition, pups were pooled and randomized into separate litters (n=17 pups/litter), placed into separate shoebox cages inside oxygen delivery chamber, and subjected to an oxygen-exposure profile from Day 0 to Day 14 postpartum. Litters were then placed into room air from Day 14/0 through Day 14/6 (days 14-20 postpartum). Additionally on Day 14/0, each pup was randomly assigned as oxygen-exposed controls, vehicle treated, or drug-treated at 1.5, 5, 10 mg/kg, p.o., BID. At Day 14/6 (20 days postpartum), all animals in both studies were euthanized and retina whole mounts were prepared as described in Example 1 above.

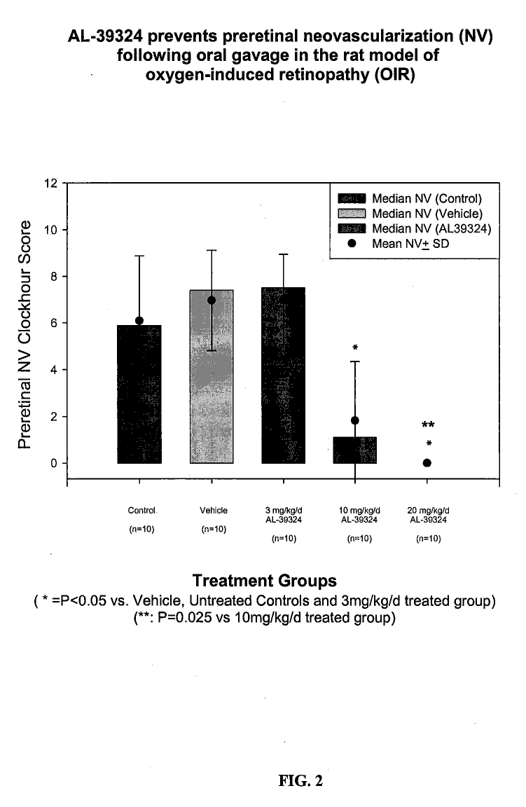

RESULTS: Systemic administration of RTKi provided potent efficacy in the rat OIR model, where 20 mg/kg/day p.o. provided complete inhibition of preretinal NV. An overall statistical difference was demonstrated between treatment groups and non-treated controls (Kruskal-Wallis one-way ANOVA test: P<0.001) (FIG. 2, Table 3). Pups receiving 10 and 20 mg/kg/day p.o. demonstrated significant inhibition of preretinal NV as compared to vehicle-treated pups, where the highest dose provided complete inhibition (Mann-Whitney rank sum test: P=0.005 and P<0.001). Pups receiving 3 mg/kg/day p.o. did not have a significant decrease in NV.

TABLE 3

| | % Inhibition | | | |
|---|---|---|---|---|
| | (vs vehicle- | | | |
| | injected | P | Median | NV |
| Treatment | eye) | value | NV | Range |
| Untreated control | | 0.427 | 5.885 | 2.5-10.5 |
| PEG 400 (vehicle) | | | 7.4 | 3.5-9.5 |
| 3 mg/kg RTKi in PEG 400 | −1.4 | 0.91 | 7.5 | 3.8-9.8 |
| 10 mg/kg RTKi in PEG 400 | 86.4 | 0.001 | 1.105 | 0-8 |
| 20 mg/kg RTKi in PEG 400 | 100 | <0.001 | 0 | 0 |

### Example 3

### Prevention of Laser-Induced Choroidal Neovascularization (CNV) Following a Intravitreal Delivery of the Receptor Kinase Tyrosine Inhibitor (RTKi), Compound 86, in the Mouse

Methods. CNV was generated by laser-induced rupture of Bruch's membrane. Briefly, 4 to 5 week old male C57BL/6J mice were anesthetized using intraperitoneal administration of ketamine hydrochloride (100 mg/kg) and xylazine (5 mg/kg) and the pupils of both eyes dilated with topical ocular instillation of 1% tropicamide and 2.5% Mydfin®. One drop of topical cellulose (Gonioscopic®) was used to lubricate the cornea. A hand-held cover slip was applied to the cornea and used as a contact lens to aid visualization of the fundus. Three to four retinal burns were placed in randomly assigned eye (right or left eye for each mouse) using the Alcon 532 nm EyeLite laser with a slit lamp delivery system. The laser burns were used to generate a rupture in Bruch's membrane, which was indicated opthalmoscopically by the formation of a bubble under the retina. Only mice with laser burns that produced three bubbles per eye were included in the study. Burns were typically placed at the 3, 6, 9 or 12 o'clock positions in the posterior pole of the retina, avoiding the branch retinal arteries and veins.

Each mouse was randomly assigned into one of the following treatment groups: noninjected controls, sham-injected controls, vehicle-injected mice, or one of three RTKi-injected groups. Control mice received laser photocoagulation in both eyes, where one eye received a sham injection, i.e. a pars plana needle puncture. For intravitreal-injected animals, one laser-treated eye received a 5 ul intravitreal injection of 0%, 0.3%, 1%, or 3% RTKI. The intravitreal injection was performed immediately after laser photocoagulation. At 14 days post-laser, all mice were anesthetized and systemically perfused with fluorescein-labeled dextran. Eyes were then harvested and prepared as choroidal flat mounts with the RPE side oriented towards the observer. All choroidal flat mounts were examined using a fluorescent microscope. Digital images of the CNV were captured, where the CNV was identified as areas of hyperfluorescence within the pigmented background. Computerized image analysis was used to delineate and measure the two dimensional area of the hyperfluorescent CNV per lesion (um²) for the outcome measurement. The median CNV area/burn per mouse per treatment group or the mean CNV area/burn per treatment group was used for statistical analysis depending on the normality of data distribution; P≦0.05 was considered significant.

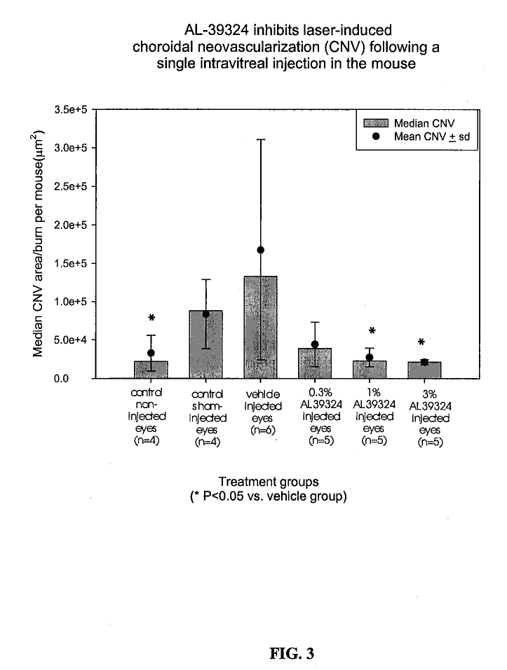

Results. Local administration of RTKi provided potent antiangiogenic efficacy in a mouse model of laser-induced CNV. An overall significant difference between treatment groups was established with a Kiruskal-Wallis one way ANOVA (P=0.015) (FIG. 3). Moreover, eyes injected with 1% RTKi (↓84.1%) and 3% RTKi (↓83.0%) showed significant inhibition of CNV as compared to vehicle-injected eyes (Mann-Whitney rank sum tests; P=0.004, and P=0.017, respectively). A marginal statistical difference was found between eyes injected with 0.3% RTKi and vehicle injected eyes (P=0.082).

The median and mean ±s.d. CNV area/burn per mouse in control groups with no injection was 21721 um² and 32612±23131 um² (n=4 mice), and with sham injection was 87854 um² and 83524±45144 um² (n=4 mice). The median and mean ±s.d. CNV area/burn per mouse in vehicle-treated mice was 133014 um² and 167330±143201 um² (n=6 mice). The median/mean ±s.d. in the 0.3%, 1% and 3% RTKi treated groups were 38891 um² and 44283±28886 um² (n=5 mice); 21122 um² and 21036±3100 um² (n=5 mice); 22665 um² and 27288±12109 um² (n=5 mice), respectively.

### Example 4

### Intravitreal Delivery of the RTKi, Compound 86, Induces Regression of Existing Laser-Induced Choroidal Neovascularization (CNV) in the Mouse

METHODS: CNV was generated by laser-induced rupture of Bruch's membrane as described above in Example 3. Each mouse was randomly assigned to one of the following treatment groups: noninjected controls, sham-injected controls, vehicle-injected mice, RTKi injected groups. Control mice received laser photocoagulation in both eyes, where one eye received a sham injection, i.e. a pars plana needle puncture. For intravitreal-injected animals, one laser-treated eye received a 5 μl intravitreal injection of 0%, 1% or 3% RTKi or 2 μl 1% RTKi. All mice received laser photocoagulation at day 0. For mice randomized to an injection group, a single intravitreal injection was performed at 7 days post-laser. Also at 7 days post-laser, several mice with no-injection were euthanized and their eyes used for controls. At 14 days post-laser, all remaining mice were euthanized and systemically perfused with fluorescein-labeled dextran. Eyes were then harvested and prepared as choroidal flat mounts with the RPE side oriented towards the observer. Choroidal flat mounts were analyzed as described above in Example 3.

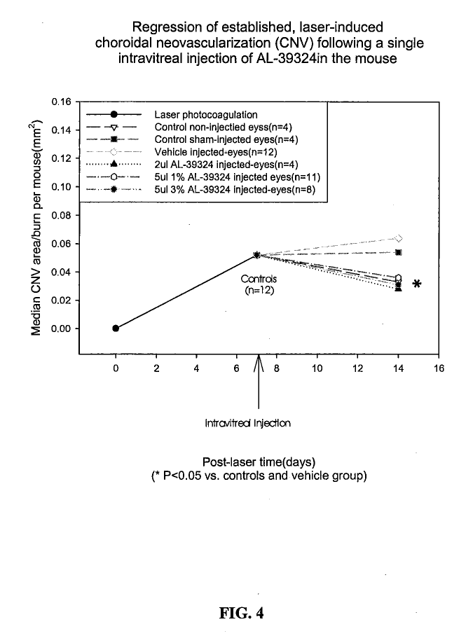

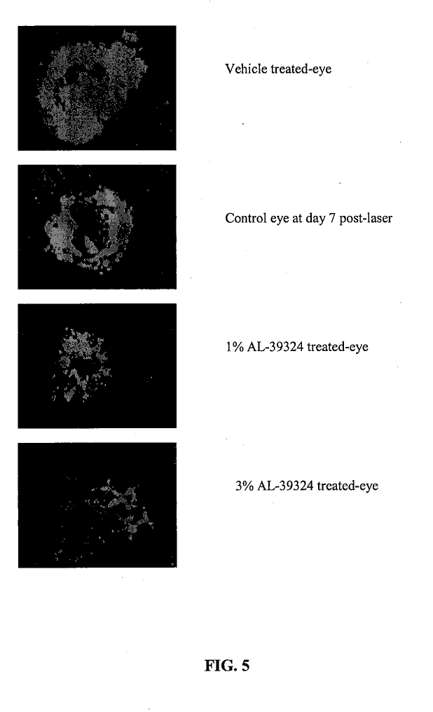

RESULTS: Local administration of RTKi caused regression of existing laser-induced CNV in the adult mouse. An overall significant difference between treatment groups was established with a Kiruskal-Wallis one way ANOVA (P=0.002) (FIG. 4). By 14 days following laser rupture of Bruch's membrane, the median CNV area in eyes injected with 2 μl 1% RTKi (↓45.4%), 5 μl 1% RTKi (↓29.7%), and 5 μl 3% RTKi (↓441.0%) was significantly reduced when compared to the amount of CNV present at 7 days post-laser (Mann-Whitney rank sum tests; P=0.025, P=0.039 and P=0.012, respectively). Eyes injected with 2 μl 1% RTKi (↓55.9%), 5 μl 1% RTKi (↓43.7%), and 3% RTKi (↓52.3%) showed significant inhibition of CNV as compared to vehicle-injected eyes at day 14 post-laser (Mann-Whitney rank sum tests; P=0.009, P=0.006, and 0.001, respectively). A gross reduction in CNV development was observed as a decrease in the hyperfluorescent area at the site of laser photocoagulation in 1% or 3% RTKi-injected eyes as compared to 1) control eyes at day 7 post-laser and 2) vehicle-injected eyes at day 14 post-laser (FIG. 5).

TABLE 4

| | Median | Mean | | |
|---|---|---|---|---|
| | CNV (μm) | CNV (μm) | SE | N (mice) |
| Control at day 7 | 51808 | 54452 | 5385 | 12 |
| Non-injected | 32881 | 34589 | 8413 | 4 |
| control at day 14 | | | | |
| Sham-injected | 54078 | 48594 | 8614 | 4 |
| control at day 14 | | | | |
| Vehicle | 64067 | 65932 | 5833 | 12 |
| 1% RTKi (2 μl) | 28268 | 30959 | 7287 | 4 |
| 1% RTKi (5 μl) | 36429 | 39178 | 5861 | 11 |
| 3% RTKi (5 μl) | 30560 | 35174 | 4110 | 8 |

### Example 5

### Systemic Administration of the RTKi, Compound 86, Provides Dose-Dependent Inhibition and Regression of Laser-Induced Choroidal Neovascularization (CNV) in the Mouse

METHODS: CNV was generated by laser-induced rupture of Bruch's membrane as described in Example 3 above. Mice were randomly assigned as oral gavage groups receiving 0, 3, 10, and 20 mg/kg/day RTKi. The mice received an oral gavage of 0, 1.5, 5, or 10 mg/kg twice per day and for 14 days post-laser. For the regression or intervention paradigm, mice were randomly assigned to groups receiving 0, 1.5, 5, or 10 mg/kg RTKi p.o. BID, (0, 3, 10, or 20 mg/kg/day) at day 7 after laser photocoagulation. Oral gavage dosing was continued twice per day for 14 days post-laser. Several mice were euthanized at day 7 post-laser and used for controls. At 14 days post-laser, all mice were anesthetized and systemically perfused with fluorescein-labeled dextran. Eyes were then harvested and prepared as choroidal flat mounts as described in Example 3 above.

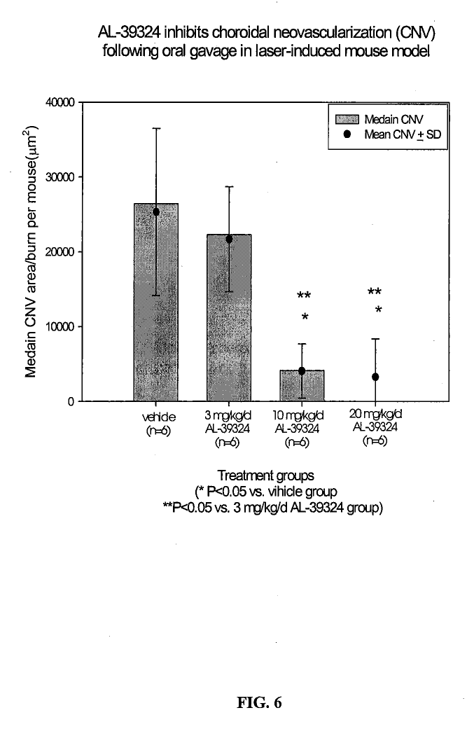

RESULTS. Systemic administration of RTKi provided potent and highly efficacious inhibition of laser-induced CNV, where mice treated 20 mg/kg/day showed complete inhibition of CNV development and significant regression of established CNV. In the prevention paradigm, an overall significant difference between treatment groups was established with a Kruskal-Wallis one way ANOVA (P<0.001) (FIG. 6, Table 5a). Moreover, systemic delivery of 20 mg/kg/d RTKi provided complete inhibition of CNV (P<0.009) and the mice treated with 10 mg/kg/day showed an 84.3% inhibition of CNV(P<0.002). Mice treated with 3 mg/kg/day exhibited no significant inhibition (P<0.589), as compared to vehicle-injected eyes (Mann-Whitney rank sum tests).

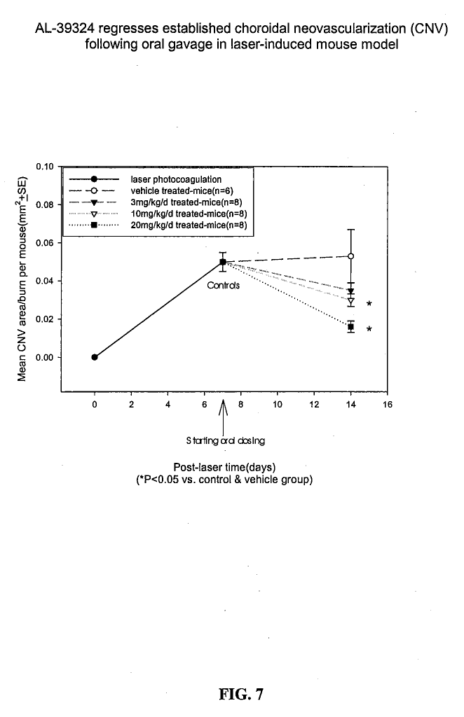

In the regression paradigm, an overall significant difference between treatment groups was established with a Kiruskal-Wallis one-way ANOVA (P<0.001) (FIG. 7 & Table 5b). Mice treated with 20 mg/kg/day and 10 mg/kg/day exhibited significant regression of existing CNV by 68.0% and 41.8%, respectively, as compared to nontreated controls (Mann-Whitney Rank Sum Test, P<0.002 and P<0.011, respectively). Mice treated with 3 mg/kg/day did not show a significant regression of existing CNV (Mann-Whitney Rank Sum Test, P>0.065). No significant difference was found between the control and vehicle treated-groups (Mann-Whitney Rank Sum Test, P=0.792).

TABLE 5a

| | Median | Mean | | |
|---|---|---|---|---|
| | CNV (μm²) | CNV (μm²) | SD | Mice number |
| Vehicle | 26417 | 25316 | 11196 | 6 |
| 3 mg/kg/day RTKi | 22317 | 21670 | 7012 | 6 |
| 10 mg/kg/day RTKi | 4137 | 4046 | 3625 | 6 |
| 20 mg/kg/day RTKi | 0 | 3266 | 5079 | 6 |

TABLE 5b

| | Median | Mean | | |
|---|---|---|---|---|
| Treatment | CNV (μm²) | CNV (μm²) | SD | Mice number |
| Control | 47055 | 49665 | 11183 | 5 |
| Vehicle | 41362 | 52974 | 33403 | 6 |
| 3 mg/kg/day RTKi | 33967 | 35442 | 11807 | 8 |
| 10 mg/kg/day RTKi | 27389 | 29773 | 9514 | 8 |
| 20 mg/kg/day RTKi | 15036 | 15706 | 8301 | 8 |

### Example 6

### Intravitreal Delivery of the RTKi, Compound 86, Inhibits VEGF-Induced Retinal Vascular Permeability in the Rat

METHODS: Adult Sprague-Dawley rats were anesthetized with intramuscular ketamine/xylazine and their pupils dilated with topical cycloplegics. Rats were randomly assigned to intravitreal injection groups of 0% 0.3%, 1.0%, and 3.0% RTKI and a positive control. Ten μl of each compound was intravitreally injected in each treatment eye (n=6 eyes per group). Three days following first intravitreal injection, all animals received an intravitreal injection of 10 μl 1400 ng hr VEGF in both eyes. Twenty-four hours post-injection of VEGF, intravenous infusion of 3% Evans blue dye was performed in all animals, where 50 mg/kg of Evans blue dye was injected via the lateral tail vein during general anesthesia. After the dye had circulated for 90 minutes, the rats were euthanized. The rats were then systemically perfused with balanced salt solution, and then both eyes of each rat were immediately enucleated and the retinas harvested using a surgical microscope. After measurement of the retinal wet weight, the Evans blue dye was extracted by placing the retina in a 0.2 ml formamide (Sigma) and then the homogenized and ultracentrifuged. Blood samples were centrifuged and the plasma diluted 100 fold in formamide. For both retina and plasma samples, 60 μl of supernatant was used to measure the Evans blue dye absorbance (ABS) with at 620/740 nm. The blood-retinal barrier breakdown and subsequent retinal vascular permeability as measured by dye absorbance were calculated as means ±s.e.m. of net ABS/wet weight/plasma ABS. A two-tailed Student's t-test were used for pair wise comparisons between OS and OD eyes in each group. One way ANOVA was used to determine an overall difference between treatment means, where P≦0.05 was considered significant.

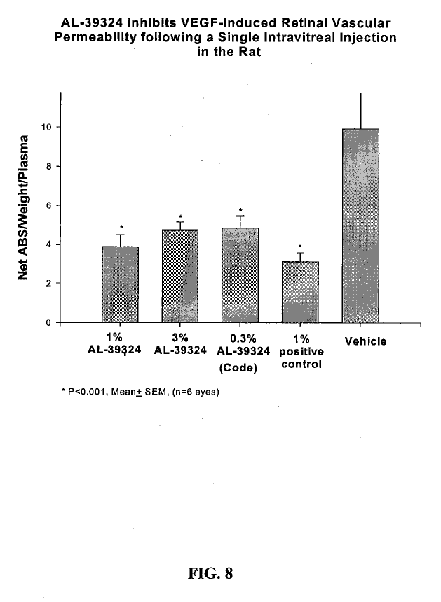

RESULTS. A single intravitreal injection of RTKi provided potent and efficacious inhibition of VEGF-induced retinal vascular permeability in the rat (FIG. 8). An overall statistical difference was demonstrated between treatment groups and vehicle controls (Student-Newman-Keuls one-way AVOVA test: P<0.001). Retinal vascular permeability was significantly decreased in eyes treated with RTK inhibitor as compared to vehicle-injected eyes: 0.3% RTKi (↓50%), 1.0% RTKi (↓61%), 3% RTKi (↓53%), and positive control (↓69%), respectively.

The mean ABS+s.e.m. in vehicle control group was 9.93±1.82. In drug treated group of 0.3% RTKi was 4.84±0.64; in 1.0% RTKi group was 3.87±0.62; in 3.0% RTKi group was 4.75±0.40 and in the positive control group was 3.11±0.46. There was no significant difference between drug treated groups.

### Example 7

### Intravitreal Delivery of the RTKi, Compound 86, Inhibits VEGF-Induced Retinal Vascular Permeability in the Rat

METHODS: Diabetes was induced in male Long-Evans rats with 65 mg/kg streptozotocin (STZ) after an overnight fast. Upon confirmation of diabetes (blood glucose >250 mg/dl), treatment was initiated by oral gavage. Non-diabetic (NDM) and diabetic (DM) rats received oral gavage of either vehicle or RTK inhibitor at 1.5 or 5 mg/kg/d BID. After 2 weeks, jugular vein catheters were implanted 1 day prior to experimentation for the infusion of indicator dye. Retinal vascular permeability, RVP, was measured using Evan's blue albumin permeation (45 mg/kg) after a 2 hour circulation period.

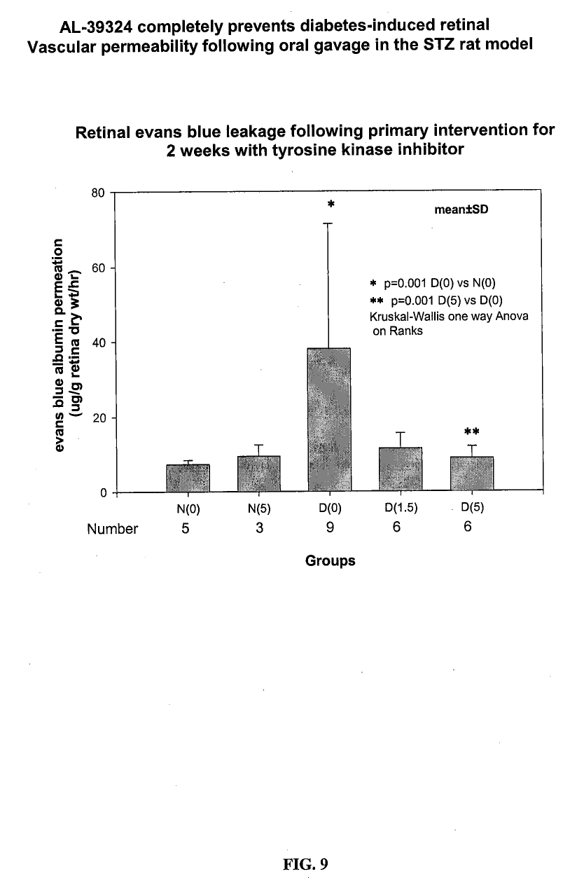

RESULTS: Treatment with the oral RTKi was well tolerated by both NDM and DM groups with no observed systemic or ERG side effects. Blood glucose levels and body weights were not different between DM control and DM treatment groups. Diabetes increased RVP (38.1±33.4 μl/g/hr, n=9) as compared with NDM control (7.3±2.5 μl/g/hr, n=5, p<0.001). RVP was significantly reduced in DM animals treated with RTKi at 1.5 mg/kg/d (11.4±4.1 μl/g/hr, n=6, p<0.05) and at 5 mg/kg/d (8.9±3.1 μl/g/hr, n=7, p<0.01) as compared to DM control (FIG. 9). RVP was unchanged in NDM treated at 5 mg/kg/d.

### Example 8

### Topical Ocular Delivery of 3-Benzoylphenylacetic Acid Inhibits Laser-Induced Choroidal Neovascularization in the Mouse

METHODS: Choroidal neovascularization was generated by modification of a previously described technique [Tobe, 1998 #448]. Briefly, 4 to 5 week old female C57BL/6J mice were anesthetized with ketamine hydrochloride (100 mg/kg body weight) and the pupils were dilated with 1% tropicamide. Three burns of 532 nm diode laser photocoagulation (75 μm spot size, 0.1 seconds duration, 120 mW) were delivered to each retina using the slit lamp delivery system of an OcuLight GL Photocoagulator (Iridex, Mountain View, Calif.) and a hand held cover slide as a contact lens. Burns were performed in the 9, 12, and 3 o'clock positions of the posterior pole of the retina. Production of a bubble at the time of laser, which indicates rupture of Bruch's membrane, is an important factor in obtaining CNV [Tobe, 1998 #448], so only burns in which a bubble was produced were included in the study. Mice were treated with one drop four times a day in masked fashion for 2 weeks and then mice were sacrificed. In some experiments, both eyes were treated with drug and these mice were compared to littermates in which both eyes were treated with vehicle. In other experiments, one eye was treated with drug and the fellow eye was treated with vehicle. Eyes were rapidly dissected for choroidal flat mounts or frozen in optimum cutting temperature embedding compound (OCT; Miles Diagnostics, Elkhart, Ind.).

### Ouantitative Measurement of the Amount of Choroidal Neovascularization

The sizes of CNV lesions were measured in choroidal flat mounts (Edelman and Castro, 2000). Mice used for the flat mount technique were anesthetized and perfused with 1 ml of phosphate-buffered saline containing 50 mg/ml of fluorescein-labeled dextran (2×10⁶ average mw, Sigma, St. Louis, Mo.) as previously described (Tobe et al., 1998a). The eyes were removed and fixed for 1 hour in 10% phosphate-buffered formalin. The cornea and lens were removed and the entire retina was carefully dissected from the eyecup. Radial cuts (4-7, average 5) were made from the edge to the equator and the eyecup was flat mounted in Aquamount with the sclera facing down. Flat mounts were examined by fluorescence microscopy on an Axioskop microscope (Zeiss, Thornwood, N.Y.) and images were digitized using a 3 color CCD video camera (IK-TU40A, Toshiba, Tokyo, Japan) and a frame grabber. Image-Pro Plus software (Media Cybernetics, Silver Spring, Md.) was used to measure the total area of hyperfluorescence associated with each burn, corresponding to the total fibrovascular scar. The areas within each eye were averaged to give one experimental value per eye for plotting the areas in the figures.

In some mice, the eyes were rapidly removed and frozen in optimum cutting temperature embedding compound (OCT; Miles Diagnostics, Elkhart, Ind.). Ten μm frozen sections were cut through entire lesions and the sections were histochemically stained with biotinylated Griffonia simplicifolia lectin B4 (GSA, Vector Laboratories, Burlingame, Calif.) which selectively binds to vascular cells. Slides were incubated in methanol/H₂O₂ for 10 minutes at 4° C., washed with 0.05 M Tris-buffered saline, pH 7.6 (TBS), and incubated for 30 minutes in 10% normal porcine serum. Slides were incubated 2 hours at room temperature with biotinylated GSA and after rinsing with 0.05M TBS, they were incubated with avidin coupled to peroxidase (Vector Laboratories) for 45 minutes at room temperature. The slides were developed with HistoMark Red (Kirkegaard and Perry, Cabin John, Md.) to give a red reaction product and counter stained with Contrast Blue (Kirkegaard and Perry).

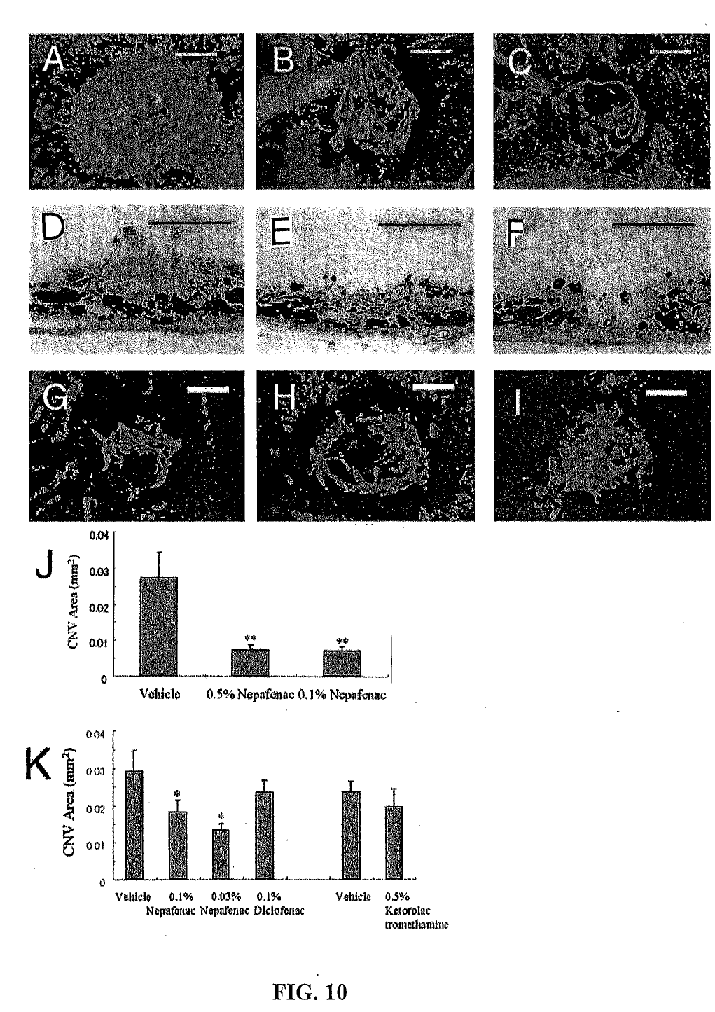

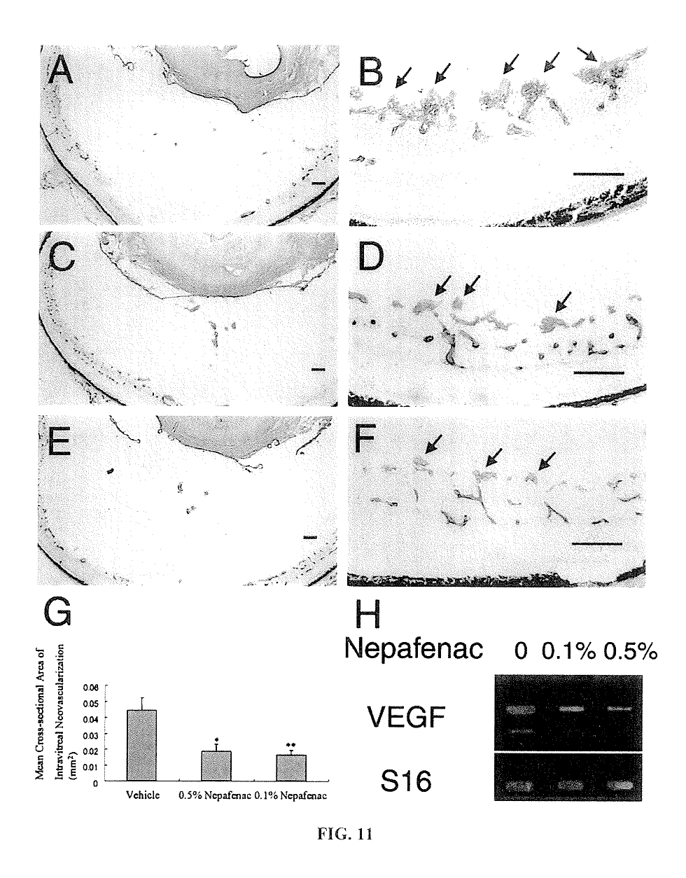

RESULTS: Mice treated with vehicle drops for 2 weeks after laser-induced rupture of Bruch's membrane developed large areas of CNV (FIG. 10A and FIG. 11D). Administration of 0.5% (FIG. 10B and FIG. 10E) or 0.1% nepafenac drops 4 times a day resulted in CNV lesions that appeared smaller and had decreased vascular density compared to the CNV in mice treated with vehicle. Measurement of CNV area in masked fashion showed that treatment with either 0.5% or 0.1% nepafenac resulted in significantly smaller CNV lesions than treatment with vehicle (FIG. 10J). In a follow-up experiment, Nepafenac was compared in masked fashion to 2 other NSAIDs, diclofenac and ketorolac tromethamine. Topical administration of 0.1% (FIG. 10G) or 0.03% (not shown) nepafanac 4 times a day for 14 days resulted CNV lesions at Bruch's membrane rupture sites that were significantly smaller than those in eyes treated with vehicle, whereas eyes treated with 0.1% diclofenac (FIG. 10H) showed no difference from vehicle-treated eyes (FIG. 10K). There was high mortality in the group of mice treated for two weeks with 0.5% ketorolac tromethamine, suggesting that this NSAID was absorbed to a greater extent into the systemic circulation and/or has more toxicity. Mice that survived 2 weeks of treatment with 0.5% ketorolac tromethamine did not appear to have less CNV than mice treated with vehicle, but numbers were insufficient for statistical comparisons. Therefore, another group of mice had laser-induced rupture of Bruch's membrane and were randomized to treatment with 0.5% ketorolac tromethamine or vehicle 4 times a day for 1 week. All mice survived and there was no difference in the size of CNV lesions between the two groups (FIG. 10K).

Although drugs were given topically, it was expected that some absorption into conjunctival blood vessels would result in drug levels in plasma that could have effects on the fellow eye. Therefore in initial studies both eyes of mice were treated with drug and comparisons were made to eyes of littermates treated with vehicle in both eyes. To explore the potential contributions of local versus systemic delivery of drug, mice with laser-induced rupture of Bruch's membrane were treated with drug in one eye and vehicle in the fellow eye. The area of CNV was significantly less in the eye treated with 0.1% Nepafenac, compared to the fellow eye. In contrast, mice treated with vehicle drops had no difference in area of CNV compared to fellow eyes, and had significantly more CNV than Nepafenac-treated eyes.

### Example 9

### Topical Ocular Delivery of 3-Benzoylphenylacetic Acid Inhibits Preretinal Neovascularizaiton in the Mouse Model of Oxygen-Induced Retinopathy

METHODS: Ischemic retinopathy was produced in C57BL/6J mice by a method described by Smith et al [Smith, 1994 #50]. Seven-day-old (P7) mice and their mothers were placed in an airtight incubator and exposed to an atmosphere of 75±3% oxygen for 5 days. Incubator temperature was maintained at 23±2° C., and oxygen was measured every 8 hours with an oxygen analyzer. After 5 days, the mice were removed from the incubator, placed in room air, and drug treatment was begun. Mice were given one drop four times a day in masked fashion. At P17, after 5 days of treatment, mice were sacrificed, eyes were rapidly removed and frozen in OCT.

Ocular frozen sections (10 μm) were histochemically stained with GSA as described above except that diaminobenzidine was used to give a brown reaction product. Slides were counterstained with eosin, which stains the internal limiting membrane and mounted with Cytoseal. To perform quantitative assessments, 10 μm serial sections were cut through the entire eye starting with sections that included the iris root on one side of the eye and proceeding to the iris root on the other side. Every tenth section, roughly 100 μm apart, were stained with GSA, examined with an Axioskop microscope, and images were digitized using a 3 CCD color video camera and a frame grabber. Image-Pro Plus software was used to delineate GSA-stained cells on the surface of the retina and their area was measured. For plotting the area measurements for the figure, the mean of the measurements from each eye was used as a single experimental value.

Mice with ischemic retinopathy were treated with 0.1% or 0.5% Nepafenac drops or vehicle drops four times a day between P12 and P17 and then sacrificed and retinal RNA was isolated using the guanidine isothiocyanate method as described by Chomczynski and Sacchi [Chomczynski, 1987 #30]. Reverse transcription was carried out with 0.5 μg of total RNA, reverse transcriptase (SuperScript II, Life Technologies, Gaithersburg, Md.), and 5.0 μM oligo d(T) primer. Aliquots of the cDNAs were used for PCR amplification with primers specific for VEGF, (5′-TTACTGCTGTACCTCCACC-3′) and (5′-ACAGGACGGCTTGAAGATG-3′). Titrations were performed to insure that PCR reactions were carried out in the linear range of amplification. Mouse S16 ribosomal protein primers (5′-CACTGCAAACGGGGAAATGG-3′ and 5′-TGAGATGGACTGTCGGATGG-3′) were used to provide an internal control for the amount of template in the PCR reactions.

RESULTS: Mice with oxygen-induced ischemic retinopathy treated with vehicle drops 4 times a day between P12 and P17 had extensive neovascularization anterior to the internal limiting membrane of the retina (FIG. 11A and FIG. 11B, arrows). Treatment 4 times a day with 0.5% (FIG. 11C and FIG. 11D) or 0.1% (FIG. 11E and FIG. 11F) nepafenac drops resulted in significantly less retinal neovascularization (FIG. 11G). Retinal RNA was isolated from nepafenac- or vehicle-treated eyes of mice with ischemic retinopathy and semi-quantitative RT-PCR was done using primers specific for VEGF. The retinas from eyes treated with 0.1% or 0.5% Nepafenac drops had less VEGF mRNA than retinas from eyes treated with vehicle (FIG. 11H).

### Example 10

### Topical Ocular Delivery of 3-Benzoylphenylacetic Acid Inhibits Pathologic Changes of the Retinal Vasculature and Neuronal Function after 9 Months of Diabetes in the STZ-Diabetic Rat

Methods. Streptozotocin (STZ)-induced diabetic rats were assigned to 3 groups (untreated control, Nepafenac eyedrops, and vehicle eyedrops) for comparison to nondiabetic controls. Eyedrops were administered in both eyes 4 times/day using both subacute (2 mos) or chronic dosing paradigms (9 mos). During the 2 month study, prostaglandin E2 (PGE₂), cyclooxygenase (COX)-2, and VEGF were measured by immunoassay, superoxide generation was measured by bioluminescence, activities of caspases 3 and 6 were measured fluorimetrically, and nitric oxide (NO) was estimated from levels of nitrite plus nitrate. Moreover, retinal leukostasis was quantified after intravenous perfusion with FITC-concanavalin A. During the 9-month study, vascular lesions associated with diabetic retinopathy were quantitated after isolation of the retinal vasculature by the trypsin digest technique, and loss of cells in the ganglion cell layer was evaluated in retinal cross-sections. In the chronic dosing study, retinal function also was assessed by measuring electroretinograms (ERGs) under dark and light adapted conditions to evaluate rod and cone pathways, respectively. The impact of topical ocular administration on corneal protease activity was assessed with MMP mRNA analysis and zymography. The signals were imported into Matlab™ (Mathworks Inc.) for further processing and the oscillatory potentials (OP's) were isolated using a 9 pole Butterworth filter with 34 Hz cutoff frequency (unity gain above 40 Hz and >12 dB attenuation 0 to 30 Hz). To simplify the analysis of OP timing the summed latency was calculated for OP's 1-4 similar to Hancock and Kraft, where TTP=(t₁+t₂+t₃+t₄) and tₓ=the time to peak of OPx.

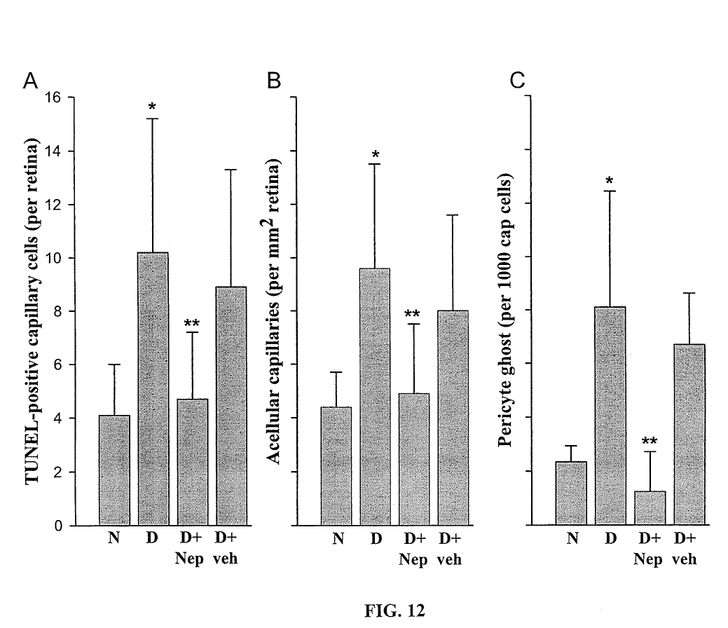

Results. After 2 months of diabetes, insulin-deficient diabetic control rats exhibited significant increases in retinal PGE₂, superoxide, expression of COX-2, and microvascular leukostasis within retinal vessels. Diabetes-induced activation of caspases 3 and 6 in retina was partially inhibited by Nepafenac, but the drug had no effect on retinal VEGF or NO levels. In the chronic dosing paradigm, a significant increase in the number of TUNEL-positive capillary cells, acellular capillaries, and pericyte ghosts was measured in diabetic rats versus nondiabetic controls. Notably, topical administration of Nepafenac significantly inhibited all of these retinal abnormalities in diabetic rats. Topical treatment with 0.3% Nepafenac completely protected the STZ-induced diabetic animals against the OP delays. Vehicle treated diabetic animals demonstrated significant prolonged TTP (223.98 msec) compared to Nepafenac-treated animals. Nepafenac-treated diabetic animals were statistically no different than nondiabetic control animals. No significant changes in corneal protease activity were measured in animal treated with topical nepafenac versus vehicle after 9 months of diabetes. (FIG. 12)

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

### U.S. Patents

U.S. Pat. No. 4,045,576
U.S. Pat. No. 4,126,635
U.S. Pat. No. 4,254,146
U.S. Pat. No. 4,313,949
U.S. Pat. No. 4,503,073
U.S. Pat. No. 4,568,695
U.S. Pat. No. 4,683,242
U.S. Pat. No. 4,910,225
U.S. Pat. No. 5,475,034
U.S. Pat. No. 6,066,671
UK application no. 2071086A
UK application no. 2093027A

### Other Publications

http://www.cellsignal.com/retail/
Adamis A P, Shima D T, Tolentino M J, et al. Inhibition of vascular endothelial growth factor prevents retinal ischemia-associated iris neovascularization in a nonhuman primate. Arch Opthalmol. 1996; 114:66-71.
Armstrong S A, Kung A L, Mabon M E, et al. Inhibition of FLT3 in MLL. Validation of a therapeutic target identified by gene expression based classification. Canc Cell. 2003; 3:173-83.
Asahara T, Chen D, Takahashi T, et al. Tie2 receptor ligands, angiopoietin-1 and angiopoietin-2, modulate VEGF-induced postnatal neovascularization. Circ Res. 1998; 83:233-40.
Benjamin Le, Hemo I, Keshet E. A plasticity window for blood vessel remodelling is defined by pericyte coverage of the preformed endothelial network and is regulated by PDGF-B and VEGF. Development. 1998; 125:1591-8.
Bergers G, Song S, Meyer-Morse N, Bergsland E, Hanahan D. Benefits of targeting both pericytes and endothelial cells in the tumor vasculature with kinase inhibitors. J Clin Inv. 2003; 111: 1287-95.
Bilodeau M T, Fraley M E, Hartman G D. Kinase insert domain-containing receptor kinase inhibitors as anti-angiogenic agents. Expert Opin Investig Drugs. 2002; 11(6):737-45.
Blume-Jensen P, Hunter T. Oncogenic kinase signalling. Nature. 2001; 411:355-65.
Boyer S J. Small molecule inhibitors of KDR (VEGFR-2) kinase: An overview of structure activity relationships. Curr Top Med. Chem. 2002; 2:973-1000.
Campochiaro P A, the C99-PKC412-003 Study Group. Reduction of diabetic macular edema by oral administration of the kinase inhibitor PKC412. IOVS. 2004; 45:922-31.
Carmeliet P, Rerreira V, Breier G, et al. Abnormal blood vessel development and lethality in embryos lacking a single VEGF allele. Nature. 1996; 380:435-9.
Chen Y-S, Hackett S F, Schoenfeld C-L, Vinores M A, Vinores S A, Campochiaro P A. Localisation of vascular endothelial growth factor and its receptors to cells of vascular and avascular epiretinal membranes. Br J Opthalmol. 1997; 81:919-26.
Chomczynski P, Sacchi N. Single-step method of RNA isolation by acid guanidinium thiocyanate-phenol-chloroform extraction. Anal. Biochem. 1987; 162:156-159.
Cousins S W, Espinosa-Heidmann D G, Csaky K G. Monocyte activation in patients with age-related macular degeneration—A biomarker of risk for choroidal neovascularization? Arch Opthalmol. 2004; 122(7): 1013-8.
Csaky K G, Baffi J Z, Byrnes G A, et al. Recruitment of marrow-derived endothelial cells to experimental choroidal neovascularization by local expression of vascular endothelial growth factor. Exp Eye Res. 2004; 78:1107-16.
Curtin M L, Frey R R, Heyman R, et al. Isoindolinone ureas: a novel class of KDR kinase inhibitors. Bioorg Med Chem Lett. 2004; 14:4505-9.
De Vries C, Escobedo J A, Ueno H, Houck K, Ferrar N, Williams L T. The fms-like tyrosine kinase, a receptor for vascular endothelial growth factor. Science. 1992; 255:989-91.
Dehmel U, Zaborski M, Meierhoff G, et al. Effects of FLT3 ligand on human leukemia cells. I. Proliferative response of myeloid leukemia cells. Leukemia. 1996; 10:261-70.
Edelman J L, Castro M R. Quantitative image analysis of laser-induced choroidal neovascularization in rat. Exp Eye Res 2000; 71:523-533.
Eriksson U, Alitalo K. VEGF receptor-1 stimulates stem-cell recruitment and new hope for angiogenesis therapies. Nat. Med. 2002; 8:775-7.
Espinosa-Heidmann D G, Caicedo A, Hernandez E P, Csaky K G, Cousins S W. Bone marrow-derived progenitor cells contribute to experimental choroidal neovascularization. IOVS. 2003; 44(11):4914-19.
Eyetech Study Group. Preclinical and phase 1A clinical evaluation of an anti-VEGF pegylated aptamer (EYE001) for the treatment of exudative age-related macular degeneration. Retina. 2002; 22:143-52.
Ferrara N, Davis-Smyth T. The biology of vascular endothelial growth factor. Endo Rev. 1997; 18:4-25
Ferrara N, Gerber H P, LeCouter J. The biology of VEGF and its receptors. Nat. Med. 2003; 9:669-76.
Fong G H, Rossant J, Gertsenstein M, Breitman M L. Role of the Flt-1 receptor tyrosine kinase in regulating the assembly of vascular endothelium. Nature. 1995; 376:66-70.
Fukumura D, Xavier R, Sugiura T, et al. Tumor induction of VEGF promoter activity in stromal cells. Cell. 1998; 94:715-25.
George D, Platelet-derived growth factor receptors: A therapeutic target in solid tumors. Semin Oncol. 2001; 28:27-33.
Grant M B, May W S, Caballero S, et al. Adult hematopoietic stem cells provide functional hemangioblast activity during retinal neovascularization. Nature Med. 2002; 6(8):607-12.
Griffloen A W, Molema G. Angiogenesis: Potentials for pharmacologic intervention in the treatment of cancer, cardiovascular diseases, and chronic inflammation. Pharm Rev. 2000; 52:237-68.
Gschwind A, Fischer O M, Ullrich A. The discovery of receptor tyrosine kinases: targets for cancer therapy. Nat Rev. 2004; 4:361-70.
Hackett S F, Ozaki H, Strauss R W, et al. Angiopoietin 2 expression in the retina: Upregulation during physiologic and pathologic neovascularization. J Cell Physiol. 2000; 184:275-84.
Hammes H-P, Lin J, Wagner, et al. Angiopoietin-2 causes pericyte dropout in the normal retina: Evidence for involvement in diabetic retinopathy. Diabetes. 2004; 53:1104-10.
Hanahan D. Signaling vascular morphogenesis and maintenance. Science. 1997; 277:48-50.
Hartnett M E, Lappas A, Darland D, McColm J R, Lovejoy S, D'Amore P A. Retinal pigment epithelium and endothelial cell interaction causes retinal pigment epithelial barrier disfunction via a soluble VEGF-dependent mechanism. Exp Eye Res. 2003; 77:593-9.
Heinrich M C, Blanke C D, Druker B J, Corless C L. Inhibition of KIT tyrosine kinase activity: A novel molecular approach to the treatment of KIT-positive malignancies. J Clin Oncol. 2002; 20: 1692-1703.
Hellström M, Kalén M, Lindahl P, Abramsson A, Betsholtz C. Role of PDGF-B and PDGFR-beta in recruitment of vascular smooth muscle cells and pericytes during embryonic blood vessel formation in the mouse. Development. 1999; 126:3047-55.
Hunter T. Signaling-100 and beyond. Cell. 2000; 100: 113-127.
Inoue M, Hager J H, Ferrara N, Gerber H P, Hanahan D. VEGF-A has a critical, nonredundant role in angiogenic switching and pancreatic beta cell carcinogenesis. Cancer Cell. 2002; 1:193-202.
Ishida S, Usui T, Yamashiro K, Kaji Y, Ahmed E, Carrasquillo K G, Amano S, Hida T, Oguchi Y, Adamis A P. VEGF₁₆₄ is proinflammatory in the diabetic retina. IOVS. 2003; 44(5):2155-62.
Ishida S, Usui T, Yamashiro K, et al. VEGF₁₆₄-mediated inflammation is required for pathological, but not physiological, ischemia-induced retinal neovascularization. J Exp Med. 2003; 198(3):483-9.
Keck P J, Hauser S D, Krivi G, Sanzo K, Warren T, Feder J, Connolly. Vascular permeability factor, an endothelial cell mitogen related to PDGF. Science. 1989; 246:1309-12.
Kiyoi H, Naoe T. FLT3 in human hematologic malignancies. Leukemia Lymphoma. 2002; 43:1541-7.
Kottaridis P D, Gale R E, Linch D C. Flt3 mutations and leukaemia. Br J Haem. 2003; 122:523-38.
Krishnan J, Kirkin V, Steffen A, et al. Differential in vivo and in vitro expression of vascular endothelial growth factor (VEGF)-C and VEGF-D in tumors and its relationship to lymphatic metastasis in immunocompetent rats. Cancer Res. 2003; 63:713-22.
Krzystolik M G, Afshari M A, Adamis A P, et al. Prevention of experimental choroidal neovascularization with intravitreal anti-vascular endothelial growth factor antibody fragment. Arch Opthalmol. 2002; 120:338-46.
Kvanta A, Algvere P V, Berglin L, Seregard S. Subfoveal fibrovascular membranes in age-related macular degeneration express vascular endothelial growth factor. IOVS. 1996; 37(9): 1929-34.
Kwak N, Okamoto N, Wood J M, Campochiaro P A. VEGF is major stimulator in model of choroidal neovascularization. IOVS. 200; 41:3158-64.
Lawrence D S, Niu J. Protein kinase inhibitors: The tyrosine-specific protein kinases. Pharmacol Ther. 1998; 77(2):81-114.
Leung D W, Cachianes G, Kuang W-J, Goeddel D V, Ferrara N. Vascular endothelial growth factor is a secreted angiogenic mitogen. Science. 1989; 246:1306-9.
Levis M, Small D. FLT3: It does matter in leukemia. Leukemia. 2003; 17:1738-52.
Lindahl P, Johansson B R, Leveen P, Betsholtz C. Pericyte loss and microaneurysm formation in PDGF-B-deficient mice. Science. 1997; 277:242-5.
Lutty G A, McLeod D S, Merges C, Diggs A, Plouet J. Localization of vascular endothelial growth factor in human retina and choroid. Arch Opthalmol. 1996; 114:971-7.
Lyden D, Hattor K, Dias S, et al. Impaired recruitment of bone-marrow-derived endothelial and hematopoietic precursor cells blocks tumor angiogenesis and growth. Nat. Med. 2001; 7:1194-1201.
Manley P W, Furet P, Bold G. Anthranilic acid amides: A novel class of antiangiogenic VEGF receptor kinase inhibitors. J Med. Chem. 2002; 45:5687-93.
Manning G, Whyte D B, Martinez R, Hunter T, Sudarsanam S. The protein kinase complement of the human genome. Sciencel 2002; 298:1912-34.
McMahon G. Presentation given at the 1^{st} International Symposium on Signal Transduction Modifiers in Cancer Therapy; Sep. 23, 2002. Amsterdam, NL.
Millauer B, Wizigmann-Voos S, Schnurch H, Martinez R, Moller N P, Risau W, Ullrich A. High affinity VEGF binding and developmental expression suggest Flk-1 as a major regulator of vasculogenesis and angiogenesis. Cell. 1993; 72:835-46.
Miller J W, Adamis A P, Shima D T, et al. Vascular endothelial growth factor/vascular permeability factor is temporally and spatially correlated with ocular angiogenesis in a primate model. Am J Pathol. 1994; 145(3)574-84.
Mudhar H S, Pollock R A, Wang C, Stiles C D, Richardson W D. PDGF and its receptors in the developing rodent retina and optic nerve. Development. 1993; 118:539-52.
Murukata C, Kaneko M, Gessner G, et al. Mixed lineage kinase activity of indolocarbazole analogues. Bioorg Med Chem Let. 2002; 12:147-50.
Nakao M, Yokota S, Iwai T, et al. Internal tandem duplication of the flt3 gene found in acute myeloid leukemia. Leukemia. 1996; 10: 1911-8.
Natali P G, Nicotra M R, Sures I, Santoro E, Bigotti A, Ullrich A. Expression of c-kit receptro in normal and transformed human nonlymphoid tissues. Cancer Res. 1992; 52:6139-43.
Oh H, Takagi H, Suzuma K, Otani A, Matsumura M, Honda Y. Hypoxia and vascular endothelial growth factor selectively up-regulate angiopoietin-2 in bovine microvascular endothelial cells. J Bio Chem. 1999; 274(22):15732-9.
Ohashi H, Takagi H, Koyama S, et al. Alterations in expression of angiopoietins and the Tie-2 receptor in the retina of streptozotocin induced diabetic rats. Mol. Vis. 2004; 10:608-17.
Ostman A, Heldin C H. Involvement of platelet-derived growth factor in disease: Development of specific antagonists. Adv Cancer Res. 2001; 20:1-38.
Otani A, Takagi H, Oh H, Koyama S, Matsumura M, Honda Y. Expressions of angiopoietins and Tie2 in human choroidal neovascular membranes. IOVS. 1999; 40(9)1912-20.
Ozaki H, Seo M-S, Ozaki K, et al. Blockade of vascular endothelial cell growth factor receptor signaling is sufficient to completely prevent retinal neovascularization. Am J Pathol. 200; 156(2)697-707.
Pietras K, Rubin K, Sjoblom T, et al. Inhibition of PDGF receptor signaling in tumor stroma enhances antitumor effect of chemotherapy. Cancer Res. 2002; 62:5476-84.
Ponten F, Ren Z, Nister M, Westermark B, Ponten J. Epithelial-stromal interactions in basal cell cancer: the PDGF system. J Inv Derm. 1994; 102:304-9
Quinn T P, Peters K G, de Vries C, Ferrara N, Williams L T. Fetal liver kinase 1 is a receptor for vascular endothelial growth factor and is selectively expressed in vascular endothelium. Proc Natl Acad Sci. 1993; 90:7533-7.
Rafii S, Lyden D, Benezra R, Hattori K, Heissig B. Vascular and haematopoietic stem cells: Novel targets for anti-angiogenesis therapy? Nat Rev Cancer. 2002; 2:826-35.
Rak J W, St Croix B D, Kerbel R S. Consequences of angiogenesis for tumor progression, metastasis and cancer therapy. Anti-Cancer Drugs. 1995; 6:3-18.
Reinmuth N, Liu W, Jung Y D, et al. Induction of VEGF in perivascular cells defines a potential paracrine mechanism for endothelial cell survival. FASEB J. 2001; 15:1239-41.
Robinson D R, Wu Y M, Lin S F. The protein tyrosine kinase family of the human genome. Oncogene. 2000; 19:5548-57.
Rosnet O, Buhring H J, deLapeyriere O, et al. Expression and signal transduction of the FLT3 tyrosine kinase receptor. Acta Haem. 1996; 95:218-23.
Rosnet O, Schiff C, Pebusque M J, et al. Human FLT3/FLK2 gene: cDNA cloning and expression in hematopoietic cells. Blood. 1993; 82:1110-9.
Saishin Y, Saishin Y, Takahashi K, Silva R L E, Hylton D, Rudge J S, Wiegand S J, Campochiaro P A. VEGF-TRAPR_{R1R2} suppresses choroidal neovascularization and VEGF-induced breakdown of the blood-retinal barrier. J Cell Physiol. 2003; 195:241-8.
Sarlos S, Rizkalla B, Moravski C J, Cao Z, Cooper M E, Wilkinson-Berka J L. Retinal angiogenesis is mediated by an interaction between the angiotensin type 2 receptor, VEGF, and angiopoietin. Am J Pathol. 2003; 163(3):879-87.
Sawyers C L. Finding the next Gleevec: FLT3 targeted kinase inhibitor therapy for acute myeloid leukemia. Canc Cell. 2002; 1:413-5.
Schlessinger J. Cell signaling by receptor tyrosine kinases. Cell. 2000; 103:211-25.
Seo M S, Kwak N, Ozaki H, et al. Dramatic inhibition of retinal and choroidal neovascularization by oral administration of a kinase inhibitor. Am J Pathol. 199; 154(6):1743-53.
Shaheen R M, Tseng W W, Davis D W, et al. Tyrosine kinase inhibition of multiple angiogenic growth factor receptors improves survival in mice bearing colon cancer liver metastases by inhibition of endothelial cell survival mechanisms. Canc Res. 2001; 61:1464-8.
Shalaby F, Rossant J, Yamaguchi T P, Gertsenstein M, Wu X F, Breitman M L, Schuh. Failure of blood-island formation and vasculogenesis in Flk-1-deficient mice. Nature. 1995; 376:62-66.
Shen W Y, Yu M J T, Barry C J, Constable I J, Rakoczy P E. Expression of cell adhesion molecules and vascular endothelial growth factor in experimental choroidal neovascularisation in the rat. Br J Opthalmol. 1998; 82:1063-71.
Sherr C J, Rettenmier C W, Sacca R, Roussel M F, Look A T, Stanley E R. The c-fms proto-oncogene product is related to the receptor for the mononuclear phagocyte growth factor, CSF-1. Cell. 1985; 41:665-76.
Shima D T, Adamis A P, Ferrara N, Yeo K-T, Yeo T-K, Allende R, Folkman J, D'Amore P A. Hypoxic induction of endothelial cell growth factors in retinal cells: Identification and characterization of vascular endothelial growth factor (VEGF) as the mitogen. Mol. Med. 1995; 1(2):182-93.
Skobe M, Fusenig N E. Tumorigenic conversion of immortal human keratinocytes through stromal cell activation. Proc Natl Acad. Sci. 1998; 95:1050-5.
Sorbera L A, Leeson P A, Bayes M. Ranibizumab. Drugs Future. 2003; 28(6):541-5.
Stirewalt D L, Radich J P. The role of FLT3 in haematopoietic malignancies. Nat Rev Cancer. 2003; 3:650-65.
Stone J, Itin A, Alon T, Pe'er J, Gnessin H, Chan-Ling T, Keshet E. Development of retinal vasculature is mediated by hypoxia-induced vascular endothelial growth factor (VEGF) expression by neuroglia. J Neurosci. 1995; 15(7):4738-47.
Takagi H, Koyama S, Seike H, et al. Potential role of the angiopoietin/Tie2 system in ischemia-induced retinal neovascularization. IOVS. 2003; 44(1):393-402.
Takahashi K, Saishin Y, Saishin Y, Mori K, Ando A, Yamamoto S, Oshima Y, Nambu H, Melia M B, Bingaman D P, Campochiaro P A. Topical nepafeac inhibits ocular neovascularization. IOVS 2003; 44(1):409-415.
Terman B I, Dougher-Vermazen M, Carrion M E, Dimitrov D, Armellino D C, Gospodarowicz D, Bohlen P. Identification of the KDR tyrosine kinase as a receptor for vascular endothelial cell growth factor. Biochem Biophys Res Comm. 1992; 187:1579-86.
Tian Q, Frierson H F Jr, Krystal G W, Moskaluk C A. Activating c-kit gene mutations in human germ cell tumors. Am J Pathol. 1999; 154:1643-7.
Tobe T, Okamoto N, Vinores M A, et al. Evolution of neovascularization in mice with overexpression of vascular endothelial growth factor in photoreceptors. Invest Opthalmol Vis Sci 1998; 39:180-188.
Tobe T, Ortega S, Luna L, et al. Targeted disruption of the FGF2 gene does not prevent choroidal neovascularization in a murine model. Am J Pathol 1998; 153:1641-1646.
Tolentino M J, Miller J W, Gragoudas E S, et al. Vascular endothelial growth factor is sufficient to produce iris neovascularization and neovascular glaucoma in a nonhuman primate. Arch Opthalmol. 1996; 114:964-70.
Tolentino M J, Miller J W, Gragoudas E S, et al. Intravitreous injections of vascular endothelial growth factor produce retinal ischemia and microangiopathy in an adult primate. Opthalmol. 1996; 103:1820-8.
Traxler P, Bold G, Buchdunger E, Caravatti G, et al. Tyrosine kinase inhibitors: From rational design to clinical trials. Med Res Rev. 2001; 21(6):499-512.
Turner A M, Zsebo K M, Martin F, Jacobsen F W, Bennett L C, Broudy V C. Nonhematopoietic tumor cell lines express stem cell factor and display c-kit receptors. Blood. 1992; 80:374-81.
Unsoeld A S, Junker B, Mazitschek R, et al. Local injection of receptor tyrosine kinase inhibitor MAE 87 reduces retinal neovascularization in mice. Mol. Vis. 2004; 10:468-75.
Walsh et al., J. MED. CHEM. 33:2296-2304 (1990).
Waltenberger J, Claesson-Welsh L, Siegbahn A, Shibuya M, Heldin C H. Different signal transduction properties of KDR and Flt1, two receptors for vascular endothelial growth factor. J Bio Chem. 1994; 269:26988-95.
Wang D, Huang H J, Kazlauskas A, Cavenee W K. Induction of vascular endothelial growth factor expression in endothelial cells by platelet-derived growth factor through the activation of phosphatidylinositol 3-kinase. Cancer Res. 1999; 59:1464-72.
Werdich X Q, McCollum G W, Rajaratnam V S, Penn J S. Variable oxygen and retinal VEGF levels: correlation with incidence and severity of pathology in a rat model of oxygen-induced retinopathy. Exp Eye Res. 2004; 79:623-30.
Wiesmann C, Fuh G, Christinger H W, EigenbrotC, Wells J A, de Vos, A M. Crystal structure at 1.7 Å resolution of VEGF in complex with domain-2 of the Flt-1 receptor. Cell. 1997; 91:695-704.
Wilkinson-Berka J L, Babic S, De-Gooyer T, et al. Inhibition of platelet-derived growth factor promotes pericyte loss and angiogenesis in ischemic retinopathy. Am J Pathol. 2004; 164(4):1263-73.
Witmer A N, Blaauwgeers H G, Weich H A, Alitalo K, Vrensen G F J M, Schlingemann R O. Altered expression patterns of VEGF receptors in human diabetic retina and in experimental VEGF-induced retinopathy in monkey. IOVS. 2002; 43(3):849-57.
Yancopoulos G D, Davis S, Gale N R, Rudge J S, Wiegand S J, Holash J. Vascular-specific growth factors and blood vessel formation. Nature. 2000; 407:242-8.

We claim:

1. A method for treating diabetic macular edema and/or ocular angiogenesis in a patient, said method comprising administering to said patient a therapeutically effective amount of an anti-inflammatory compound and a therapeutically effective amount of an active agent selected from the group consisting of anti-VEGF molecules and receptor tyrosine kinase (RTK) inhibitors, wherein the RTK inhibitor blocks tyrosine autophosphorylation of VEGFR-1, VEGFR-2, VEGFR-3, Tie-2, PDGFR, c-KIT, Flt-3, and CSF-1R.

2. The method of claim 1, wherein the active agent is a RTK inhibitor.

3. The method of claim 2, wherein the RTK inhibitor has an IC₅₀ of from 0.1 nM to 250 nM for each of the receptors listed in claim 1.

4. The method of claim 3, wherein the RTK inhibitor blocks tyrosine autophosphorylation of Tie-2, PDGFR, and VEGF receptor 2 with an IC₅₀ of from 0.1 nM to 200 nM for each receptor.

5. The method of claim 3, wherein the RTK inhibitor has an IC₅₀ of from 0.1 nM to 100 nM for at least six of the receptor listed in claim 1.

6. The method of claim 5, wherein the RTK inhibitor has an IC₅₀ of from 0.1 nM to 10 nM for at least four of the receptors listed in claim 1.

7. The method of claim 2, wherein the RTK inhibitor blocks tyrosine autophosphorylation of VEGF receptor 2, VEGF receptor 1, PDGFR, and Tie-2.

8. The method of claim 7, wherein the RTK inhibitor has an IC₅₀ of from 0.1 nM to 200 nM for each of the receptors listed in claim 7.

9. The method of claim 2, wherein the RTK inhibitor blocks tyrosine autophosphorylation of VEGF receptor 2, VEGF receptor 1, and Tie-2.

10. The method of claim 9, wherein the RTK inhibitor has an IC₅₀ of from 0.1 nM to 200 nM for each of the receptors listed in claim 9.

11. The method of claim 2, wherein the RTK inhibitor blocks tyrosine autophosphorylation of VEGF receptor 2, VEGF receptor 1, and PDGFR.

12. The method of claim 11, wherein the RTK inhibitor has an IC₅₀ of from 0.1 nM to 100 nM for each of the receptors listed in claim 11.

13. The method of claim 2, wherein the RTK inhibitor blocks tyrosine autophosphorylation of VEGF receptor 2 and Tie-2.

14. The method of claim 13, wherein the RTK inhibitor has an IC₅₀ of from 0.1 nM to 200 nM for each of the receptors listed in claim 13.

15. The method of claim 14, wherein the RTK inhibitor has an IC₅₀ of less than 10 nM for at least one of the receptors listed in claim 13.

16. The method of claim 2, wherein the RTK inhibitor blocks tyrosine autophosphorylation of VEGF receptor 2 and PDGFR.

17. The method of claim 16, wherein the RTK inhibitor has an IC₅₀ of from 0.1 nM to 100 nM for each of the receptors listed in claim 16.

18. The method of claim 17, wherein the RTK inhibitor has an IC₅₀ of less than 10 nM for at least one of the receptors listed in claim 16.

19. The method of claim 2, wherein the RTK inhibitor blocks tyrosine autophosphorylation of VEGF receptor 2, Tie-2, and PDGFR.

20. The method of claim 19, wherein the RTK inhibitor has an IC₅₀ of between 0.1 nM and 200 nM for each of the receptors listed in claim 19.

21. The method of claim 20, wherein the RTK inhibitor has an IC₅₀ of less than 10 nM for at least one of the receptors listed in claim 19.

22. The method of claim 2, wherein said RTK inhibitor may be selected from the group consisting of N-[4-(3-amino-1,2-benzisoxazol-4-yl)phenyl]-N′-(3-methylphenyl)urea;
N-[4-(3-amino-1,2-benzisoxazol-4-yl)phenyl]-N′-[2-(trifluoromethyl)phenyl]urea;
N-[4-(3-amino-1,2-benzisoxazol-4-yl)phenyl]-N′-(2-fluoro-5-methylphenyl)urea;
N-[4-(3-amino-1,2-benzisoxazol-4-yl)phenyl]-N′-[3-(trifluoromethyl)phenyl]urea;
N-[4-(3-amino-1,2-benzisoxazol-4-yl)phenyl]-N′-[2-fluoro-5-(trifluoromethyl)phenyl]urea;
N-[4-(3-amino-7-methoxy-1,2-benzisoxazol-4-yl)phenyl]-N′-[2-fluoro-5-(trifluoromethyl)phenyl]urea;
N-[4-(3-amino-7-methoxy-1,2-benzisoxazol-4-yl)phenyl]-N′-(3-methylphenyl)urea;
N-[4-(3-amino-7-methoxy-1,2-benzisoxazol-4-yl)phenyl]-N′-[3-(trifluoromethyl)phenyl]urea;
N-[4-(3-amino-7-methoxy-1,2-benzisoxazol-4-yl)phenyl]-N′-(3-chlorophenyl)urea;
N-[4-(3-amino-7-methoxy-1,2-benzisoxazol-4-yl)phenyl]-N′-(2-fluoro-5-methylphenyl)urea;
N-{4-[3-amino-7-(4-morpholinylmethyl)-1,2-benzisoxazol-4-yl]phenyl}-N′-[2-fluoro-5-(trifluoromethyl)phenyl]urea;
N-{4-[3-amino-7-(4-morpholinylmethyl)-1,2-benzisoxazol-4-yl]phenyl}-N′-[3-(trifluoromethyl)phenyl]urea;
N-{4-[3-amino-7-(4-morpholinylmethyl)-1,2-benzisoxazol-4-yl]phenyl}-N′-(3-chlorophenyl)urea;
N-{4-[3-amino-7-(4-morpholinylmethyl)-1,2-benzisoxazol-4-yl]phenyl}-N′-(3-methylphenyl)urea;
N-{4-[3-amino-7-(4-morpholinylmethyl)-1,2-benzisoxazol-4-yl]phenyl}-N′-(2-fluoro-5-methylphenyl)urea;
N-{4-[3-amino-7-(4-morpholinylmethyl)-1,2-benzisoxazol-4-yl]phenyl}-N′-(3,5-dimethylphenyl)urea;
N-{4-[3-amino-7-(4-morpholinylmethyl)-1,2-benzisoxazol-4-yl]phenyl}-N′-(3-phenoxyphenyl)urea;
N-{4-[3-amino-7-(4-morpholinylmethyl)-1,2-benzisoxazol-4-yl]phenyl}-N′-(3-bromophenyl)urea;
N-(4-{3-amino-7-[2-(4-morpholinyl)ethoxy]-1,2-benzisoxazol-4-yl}phenyl)-N′-[3-(trifluoromethyl)phenyl]urea;
N-(4-{3-amino-7-[2-(4-morpholinyl)ethoxy]-1,2-benzisoxazol-4-yl}phenyl)-N′-(2-fluoro-5-methylphenyl)urea;
N-(4-{3-amino-7-[2-(4-morpholinyl)ethoxy]-1,2-benzisoxazol-4-yl}phenyl)-N′-[2-fluoro-5-(trifluoromethyl)phenyl]urea;
N-(4-{3-amino-7-[2-(4-morpholinyl)ethoxy]-1,2-benzisoxazol-4-yl}phenyl)-N′-(3-methylphenyl)urea;
N-[4-(3-amino-1,2-benzisoxazol-4-yl)phenyl]-N′-(3,5-dimethylphenyl)urea;
N-[4-(3-amino-1,2-benzisoxazol-4-yl)phenyl]-N′-phenylurea;
N-[4-(3-amino-1,2-benzisoxazol-4-yl)phenyl]-N′-(4-methylphenyl)urea;
N-[4-(3-amino-1,2-benzisoxazol-4-yl)phenyl]-N′-(3-cyanophenyl)urea;
N-[4-(3-amino-1,2-benzisoxazol-4-yl)phenyl]-N′-[4-fluoro-3-(trifluoromethyl)phenyl]urea;
N-[4-(3-amino-1,2-benzisoxazol-4-yl)phenyl]-N′-(3-bromophenyl)urea;
N-[4-(3-amino-1,2-benzisoxazol-4-yl)phenyl]-N′-(3-chlorophenyl)urea;
N-[4-(3-amino-1,2-benzisoxazol-4-yl)phenyl]-N′-(3-ethylphenyl)urea;
N-[4-(3-amino-1,2-benzisoxazol-4-yl)phenyl]-N′-[4-(trifluoromethyl)phenyl]urea;
N-[4-(3-amino-1,2-benzisoxazol-4-yl)phenyl]-N′-(3-fluoro-4-methylphenyl)urea;
N-[4-(3-amino-1,2-benzisoxazol-4-yl)phenyl]-N′-(3-fluorophenyl)urea;
N-[4-(3-amino-1,2-benzisoxazol-4-yl)phenyl]-N′-(3,5-difluorophenyl)urea;
N-[4-(3-amino-1,2-benzisoxazol-4-yl)phenyl]-N′-(3-methoxyphenyl)urea;
N-[4-(3-amino-1,2-benzisoxazol-4-yl)phenyl]-N′-(4-methoxyphenyl)urea;
N-[4-(3-amino-1,2-benzisoxazol-4-yl)phenyl]urea;
N-[4-(3-amino-1,2-benzisoxazol-4-yl)phenyl]-N′-(3-nitrophenyl)urea;
N-[4-(3-amino-1,2-benzisoxazol-4-yl)phenyl]-N′-(4-fluorophenyl)urea;
N-[4-(3-amino-1,2-benzisoxazol-4-yl)phenyl]-N′-(2-fluorophenyl)urea;
N-[4-(3-amino-1,2-benzisoxazol-4-yl)phenyl]-N′-(3-chloro-4-fluorophenyl)urea;
N-[4-(3-amino-1,2-benzisoxazol-4-yl)phenyl]-N′-(3-chloro-4-methoxyphenyl)urea;
N-[4-(3-amino-1,2-benzisoxazol-4-yl)phenyl]-N′-[4-(dimethylamino)phenyl]urea;
N-[4-(3-amino-1,2-benzisoxazol-4-yl)phenyl]-N′-[4-(trifluoromethoxy)phenyl]urea;
N-[4-(3-amino-1,2-benzisoxazol-4-yl)phenyl]-N′-[2-(trifluoromethoxy)phenyl]urea;
N-[4-(3-amino-1,2-benzisoxazol-4-yl)phenyl]-N′-[3,5-bis(trifluoromethyl)phenyl]urea;
N-[4-(3-amino-1,2-benzisoxazol-4-yl)phenyl]-N′-(3-chloro-4-methylphenyl)urea;
N-[4-(3-amino-7-methoxy-1,2-benzisoxazol-4-yl)phenyl]-N′-[3,5-bis(trifluoromethyl)phenyl]urea;
N-[4-(3-amino-7-methoxy-1,2-benzisoxazol-4-yl)phenyl]-N′-[4-(trifluoromethoxy)phenyl]urea;
N-[4-(3-amino-7-methoxy-1,2-benzisoxazol-4-yl)phenyl]-N′-(3-fluorophenyl)urea;
N-[4-(3-amino-7-methoxy-1,2-benzisoxazol-4-yl)phenyl]-N′-(3-methoxyphenyl)urea;
N-[4-(3-amino-7-methoxy-1,2-benzisoxazol-4-yl)phenyl]-N′-(3,5-difluorophenyl)urea;
N-[4-(3-amino-7-methoxy-1,2-benzisoxazol-4-yl)phenyl]-N′-(4-methylphenyl)urea;
N-[4-(3-amino-7-methoxy-1,2-benzisoxazol-4-yl)phenyl]-N′-(3-bromophenyl)urea;
N-[4-(3-amino-7-methoxy-1,2-benzisoxazol-4-yl)phenyl]-N′-(3,5-dimethylphenyl)urea;
N-[4-(3-amino-7-methoxy-1,2-benzisoxazol-4-yl)phenyl]-N′-[4-(dimethylamino)phenyl]urea;
N-[4-(3-amino-7-methyl-1,2-benzisoxazol-4-yl)phenyl]-N′-(3-methylphenyl)urea;
N-[4-(3-amino-7-methyl-1,2-benzisoxazol-4-yl)phenyl]-N′-(3-chlorophenyl)urea;
N-[4-(3-amino-7-methyl-1,2-benzisoxazol-4-yl)phenyl]-N′-(2-fluoro-5-methylphenyl)urea;
N-[4-(3-amino-7-methyl-1,2-benzisoxazol-4-yl)phenyl]-N′-[2-fluoro-5-(trifluoromethyl)phenyl]urea;
N-[4-(3-amino-7-methyl-1,2-benzisoxazol-4-yl)phenyl]-N′-[3-(trifluoromethyl)phenyl]urea;
N-[4-(3-amino-7-methyl-1,2-benzisoxazol-4-yl)phenyl]-N′-(3,5-dimethylphenyl)urea;
N-[4-(3-amino-7-methyl-1,2-benzisoxazol-4-yl)phenyl]-N′-(3-ethylphenyl)urea;
N-[4-(3-amino-7-methyl-1,2-benzisoxazol-4-yl)phenyl]-N′-(4-methylphenyl)urea;
N-[4-(3-amino-7-methyl-1,2-benzisoxazol-4-yl)phenyl]-N′-[4-(trifluoromethoxy)phenyl]urea;
N-[4-(3-amino-7-methyl-1,2-benzisoxazol-4-yl)phenyl]-N′-(3-fluoro-4-methylphenyl)urea;
N-[4-(3-amino-7-methyl-1,2-benzisoxazol-4-yl)phenyl]-N′-(3-methoxyphenyl)urea;
N-[4-(3-amino-7-methyl-1,2-benzisoxazol-4-yl)phenyl]-N′-phenylurea;
N-[4-(3-amino-7-methyl-1,2-benzisoxazol-4-yl)phenyl]-N′-[3,5-bis(trifluoromethyl)phenyl]urea;
N-[4-(3-amino-7-methyl-1,2-benzisoxazol-4-yl)phenyl]-N′-(3-bromophenyl)urea;
N-[4-(3-amino-7-methyl-1,2-benzisoxazol-4-yl)phenyl]-N′-(3-fluorophenyl)urea;
N-[4-(3-amino-7-methoxy-1,2-benzisoxazol-4-yl)phenyl]-N′-[4-fluoro-3-(trifluoromethyl)phenyl]urea;
N-[4-(3-amino-7-methoxy-1,2-benzisoxazol-4-yl)phenyl]-N′-(4-fluoro-3-methylphenyl)urea;
N-[4-(3-amino-7-fluoro-1,2-benzisoxazol-4-yl)phenyl]-N′-[3-(trifluoromethyl)phenyl]urea;
N-[4-(3-amino-7-fluoro-1,2-benzisoxazol-4-yl)phenyl]-N′-(3-chlorophenyl)urea;
N-[4-(3-amino-7-fluoro-1,2-benzisoxazol-4-yl)phenyl]-N′-[4-fluoro-3-(trifluoromethyl)phenyl]urea;
N-[4-(3-amino-7-fluoro-1,2-benzisoxazol-4-yl)phenyl]-N′-(3-methylphenyl)urea;
N-[4-(3-amino-7-fluoro-1,2-benzisoxazol-4-yl)phenyl]-N′-[2-fluoro-5-(trifluoromethyl)phenyl]urea;
N-[4-(3-amino-7-fluoro-1,2-benzisoxazol-4-yl)phenyl]-N′-(2-fluoro-5-methylphenyl)urea;
N-{4-[3-amino-7-(trifluoromethoxy)-1,2-benzisoxazol-4-yl]phenyl}-N′-[2-fluoro-5-(trifluoromethyl)phenyl]urea;
N-{4-[3-amino-7-(trifluoromethoxy)-1,2-benzisoxazol-4-yl]phenyl}-N′-[3-(trifluoromethyl)phenyl]urea;
N-{4-[3-amino-7-(trifluoromethoxy)-1,2-benzisoxazol-4-yl]phenyl}-N′-(2-fluoro-5-methylphenyl)urea;
N-{4-[3-amino-7-(trifluoromethoxy)-1,2-benzisoxazol-4-yl]phenyl}-N′-(3-chlorophenyl)urea;
N-{4-[3-amino-7-(trifluoromethoxy)-1,2-benzisoxazol-4-yl]phenyl}-N′-(3-bromophenyl)urea;
5, N-{4-[3-amino-7-(trifluoromethoxy)-1,2-benzisoxazol-4-yl]phenyl}-N′-[4-fluoro-3-(trifluoromethyl)phenyl]urea;
N-[4-[3-amino-1H-indazol-4-yl]phenyl]-N′-(2-fluoro-5-methoylphenyl)urea;
N-{4-[3-amino-7-(trifluoromethoxy)-1,2-benzisoxazol-4-yl]phenyl}-N′-(4-fluoro-3-methylphenyl)urea;
N-[4-(3-amino-1H-indazol-4-yl)phenyl]-N′-(3-methylphenyl)urea;
N-[4-(3-amino-1H-indazol-4-yl)phenyl]-N′-(3,5-dimethoxyphenyl)urea;
N-[4-(3-amino-1H-indazol-4-yl)phenyl]-N′-(3-chlorophenyl)urea;
N-[4-(3-amino-1H-indazol-4-yl)phenyl]-N′-[3-(trifluoromethyl)phenyl]urea;
N-[4-(3-amino-1H-indazol-4-yl)phenyl]-N′-[2-fluoro-5-(trifluormethyl)phenyl]urea;
N-[4-(3-amino-1H-indazol-4-yl)phenyl]-N′-(3-bromophenyl)urea;
N-[4-(3-amino-1H-indazol-4-yl)phenyl]-N′-(3-bromo-4-methylphenyl)urea;
N-[4-(3-amino-1H-indazol-4-yl)phenyl]-N′-(3-ethylphenyl)urea;
N-[4-(3-amino-1H-indazol-4-yl)phenyl]-N′-phenyl urea;
N-[4-(3-amino-1H-indazol-4-yl)phenyl]-N′-(3-fluoro-4-methylphenyl)urea;
N-[4-(3-amino-1H-indazol-4-yl)phenyl]-N′-(2-fluorophenyl)urea;
N-[4-(3-amino-1H-indazol-4-yl)phenyl]-N′-(4-fluorophenyl)urea;
N-[4-(3-amino-1H-indazol-4-yl)phenyl]-N′-(3-fluorophenyl)urea;
N-[4-(3-amino-1H-indazol-4-yl)phenyl]-N′-(3-hydroxyphenyl)urea;
N-[4-(3-amino-1H-indazol-4-yl)phenyl]-N′-(3-methylphenyl)urea;
N-[4-(3-amino-1H-indazol-4-yl)-2-fluorophenyl]-N′-(2-fluoro-5-methylphenyl)urea;
N-[4-(3-amino-1H-indazol-4-yl)phenyl]-N′-[4-fluoro-3-(trifluoromethyl)phenyl]urea;
N-[4-(3-amino-1H-indazol-4-yl)phenyl]-N′-[2-fluoro-3-(trifluoromethyl)phenyl]urea;
N-[4-(3-amino-1H-indazol-4-yl)phenyl]-N′-(4-bromo-2-fluorophenyl)urea;
N-[4-(3-amino-1H-indazol-4-yl)phenyl]-N′-(5-fluoro-2-methylphenyl)urea;
N-[4-(3-amino-1H-indazol-4-yl)phenyl]-N′-(4-fluoro-3-methylphenyl)urea;
N-[4-(3-amino-1H-indazol-4-yl)phenyl]-N′-[2-fluoro-5-(hydroxymethyl)phenyl]urea;
3-[({[4-(3-amino-1H-indazol-4-yl)phenyl]amino}carbonyl)amino]-4-fluorobenzoic acid; and
Methyl 3-[({[4-(3-amino-1H-indazol-4-yl)phenyl]amino}carbonyl)amino]-4-fluorobenzoate.

23. The method of claim 22, wherein the RTK inhibitor is N-[4-[3-amino-1H-indazol-4-yl]phenyl]-N′-(2-fluoro-5-methoylphenyl)urea.

24. The method of claim 1, wherein the anti-inflammatory compound is selected from the group consisting of a COXII inhibitor, a COX I/II inhibitor, and a glucocorticoid.

25. The method of claim 24, wherein the anti-inflammatory compound is a COXII inhibitor.

26. The method of claim 25, wherein the COX II inhibitor is a 3-benzoylphenylacetic acid derivative.

27. The method of claim 26, wherein the 3-benzoylphenylacetic acid derivative is nepafenac.

28. The method of claim 1, wherein said anti-inflammatory agent is administered topically and said RTK inhibitor is administered by a method selected from the group consisting of intravitreal injection, subTenon administration, posterior juxtascleral depot administration, and implant.

29. The method of claim 1, wherein said anti-inflammatory agent and said RTK inhibitor are administered intravitreally.

30. The method of claim 1, wherein the anti-inflammatory agent and the RTK inhibitor are present in a single composition.

31. The method of claim 30, wherein the amount of anti-inflammatory agent in the composition is from 0.01% to 10% and the amount of RTK inhibitor in the composition is from 0.0001% to 30%.

32. The method of claim 30, wherein the composition is administered by a method selected from the group consisting of intravitreal injection, posterior juxtascleral depot administration, subTenon administration, and implant.

33. The method of claim 32, wherein the composition is administered via intravitreal injection.

34. A composition for treating retinal edema and/or ocular angiogenesis, comprising a therapeutically effective amount of a receptor tyrosine kinase (RTK) inhibitor and a therapeutically effective amount of an anti-inflammatory agent, wherein the RTK inhibitor blocks tyrosine autophosphorylation of VEGFR-1, VEGFR-2, VEGFR-3, Tie-2, PDGFR, c-KIT, Flt-3, and CSF-1R.

35. The composition of claim 34, wherein the RTK inhibitor has an IC₅₀ of from 0.1 nM to 250 nM for each of the receptors listed in claim 34.

36. The composition of claim 34, wherein the RTK inhibitor blocks tyrosine autophosphorylation of Tie-2, PDGFR, and VEGFR-2 with an IC₅₀ of from 0.1 nM to 200 nM for each receptor.

37. The composition of claim 34, wherein the RTK inhibitor has an IC₅₀ of from 0.1 nM to 100 nM for at least six of the receptor listed in claim 34.

38. The composition of claim 34, wherein the RTK inhibitor has an IC₅₀ of from 0.1 nM to 10 nM for at least four of the receptors listed in claim 34.

39. The composition of claim 34, wherein the RTK inhibitor blocks tyrosine autophosphorylation of VEGFR-2, VEGFR-1, PDGFR, and Tie-2.

40. The composition of claim 39, wherein the RTK inhibitor has an IC₅₀ of from 0.1 nM to 200 nM for each of the receptors listed in claim 39.

41. The composition of claim 34, wherein the RTK inhibitor blocks tyrosine autophosphorylation of VEGFR-2, VEGFR-1, and Tie-2.

42. The composition of claim 41, wherein the RTK inhibitor has an IC₅₀ of from 0.1 nM to 200 nM for each of the receptors listed in claim 41.

43. The composition of claim 34, wherein the RTK inhibitor blocks tyrosine autophosphorylation of VEGFR-2, VEGFR-1, and PDGFR.

44. The composition of claim 43, wherein the RTK inhibitor has an IC₅₀ of from 0.1 nM to 100 nM for each of the receptors listed in claim 43.

45. The composition of claim 34, wherein the RTK inhibitor blocks tyrosine autophosphorylation of VEGFR-2 and Tie-2.

46. The composition of claim 45, wherein the RTK inhibitor has an IC₅₀ of from 0.1 nM to 200 nM for each of the receptors listed in claim 45.

47. The composition of claim 46, wherein the RTK inhibitor has an IC₅₀ of less than 10 nM for at least one of the receptors listed in claim 45.

48. The composition of claim 34, wherein the RTK inhibitor blocks tyrosine autophosphorylation of VEGFR-2 and PDGFR.

49. The composition of claim 48, wherein the RTK inhibitor has an IC₅₀ of from 0.1 nM to 100 nM for each of the receptors listed in claim 48.

50. The composition of claim 49, wherein the RTK inhibitor has an IC₅₀ of less than 10 nM for at least one of the receptors listed in claim 48.

51. The composition of claim 34, wherein the RTK inhibitor blocks tyrosine autophosphorylation of VEGFR-2, Tie-2, and PDGFR.

52. The composition of claim 51, wherein the RTK inhibitor has an IC₅₀ of between 0.1 nM and 200 nM for each of the receptors listed in claim 51.

53. The composition of claim 52, wherein the RTK inhibitor has an IC₅₀ of less than 10 nM for at least one of the receptors listed in claim 51.

54. The composition of claim 34, wherein said RTK inhibitor may be selected from the group consisting of N-[4-(3-amino-1,2-benzisoxazol-4-yl)phenyl]-N′-(3-methylphenyl)urea;
N-[4-(3-amino-1,2-benzisoxazol-4-yl)phenyl]-N′-[2-(trifluoromethyl)phenyl]urea;
N-[4-(3-amino-1,2-benzisoxazol-4-yl)phenyl]-N′-(2-fluoro-5-methylphenyl)urea;
N-[4-(3-amino-1,2-benzisoxazol-4-yl)phenyl]-N′-[3-(trifluoromethyl)phenyl]urea;
N-[4-(3-amino-1,2-benzisoxazol-4-yl)phenyl]-N′-[2-fluoro-5-(trifluoromethyl)phenyl]urea;
N-[4-(3-amino-7-methoxy-1,2-benzisoxazol-4-yl)phenyl]-N′-[2-fluoro-5-(trifluoromethyl)phenyl]urea;
N-[4-(3-amino-7-methoxy-1,2-benzisoxazol-4-yl)phenyl]-N′-(3-methylphenyl)urea;
N-[4-(3-amino-7-methoxy-1,2-benzisoxazol-4-yl)phenyl]-N′-[3-(trifluoromethyl)phenyl]urea;
N-[4-(3-amino-7-methoxy-1,2-benzisoxazol-4-yl)phenyl]-N′-(3-chlorophenyl)urea;
N-[4-(3-amino-7-methoxy-1,2-benzisoxazol-4-yl)phenyl]-N′-(2-fluoro-5-methylphenyl)urea;
N-{4-[3-amino-7-(4-morpholinylmethyl)-1,2-benzisoxazol-4-yl]phenyl}-N′-[2-fluoro-5-(trifluoromethyl)phenyl]urea;
N-{4-[3-amino-7-(4-morpholinylmethyl)-1,2-benzisoxazol-4-yl]phenyl}-N′-[3-(trifluoromethyl)phenyl]urea;
N-{4-[3-amino-7-(4-morpholinylmethyl)-1,2-benzisoxazol-4-yl]phenyl}-N′-(3-chlorophenyl)urea;
N-{4-[3-amino-7-(4-morpholinylmethyl)-1,2-benzisoxazol-4-yl]phenyl}-N′-(3-methylphenyl)urea;
N-{4-[3-amino-7-(4-morpholinylmethyl)-1,2-benzisoxazol-4-yl]phenyl}-N′-(2-fluoro-5-methylphenyl)urea;
N-{4-[3-amino-7-(4-morpholinylmethyl)-1,2-benzisoxazol-4-yl]phenyl}-N′-(3,5-dimethylphenyl)urea;
N-{4-[3-amino-7-(4-morpholinylmethyl)-1,2-benzisoxazol-4-yl]phenyl}-N′-(3-phenoxyphenyl)urea;
N-{4-[3-amino-7-(4-morpholinylmethyl)-1,2-benzisoxazol-4-yl]phenyl}-N′-(3-bromophenyl)urea;
N-(4-{3-amino-7-[2-(4-morpholinyl)ethoxy]-1,2-benzisoxazol-4-yl}phenyl)-N′-[3-(trifluoromethyl)phenyl]urea;
N-(4-{3-amino-7-[2-(4-morpholinyl)ethoxy]-1,2-benzisoxazol-4-yl}phenyl)-N′-(2-fluoro-5-methylphenyl)urea;
N-(4-{3-amino-7-[2-(4-morpholinyl)ethoxy]-1,2-benzisoxazol-4-yl}phenyl)-N′-[2-fluoro-5-(trifluoromethyl)phenyl]urea;
N-(4-{3-amino-7-[2-(4-morpholinyl)ethoxy]-1,2-benzisoxazol-4-yl}phenyl)-N′-(3-methylphenyl)urea;
N-[4-(3-amino-1,2-benzisoxazol-4-yl)phenyl]-N′-(3,5-dimethylphenyl)urea;
N-[4-(3-amino-1,2-benzisoxazol-4-yl)phenyl]-N′-phenylurea;
N-[4-(3-amino-1,2-benzisoxazol-4-yl)phenyl]-N′-(4-methylphenyl)urea;
N-[4-(3-amino-1,2-benzisoxazol-4-yl)phenyl]-N′-(3-cyanophenyl)urea;
N-[4-(3-amino-1,2-benzisoxazol-4-yl)phenyl]-N′-[4-fluoro-3-(trifluoromethyl)phenyl]urea;
N-[4-(3-amino-1,2-benzisoxazol-4-yl)phenyl]-N′-(3-bromophenyl)urea;
N-[4-(3-amino-1,2-benzisoxazol-4-yl)phenyl]-N′-(3-chlorophenyl)urea;
N-[4-(3-amino-1,2-benzisoxazol-4-yl)phenyl]-N′-(3-ethylphenyl)urea;
N-[4-(3-amino-1,2-benzisoxazol-4-yl)phenyl]-N′-[4-(trifluoromethyl)phenyl]urea;
N-[4-(3-amino-1,2-benzisoxazol-4-yl)phenyl]-N′-(3-fluoro-4-methylphenyl)urea;
N-[4-(3-amino-1,2-benzisoxazol-4-yl)phenyl]-N′-(3-fluorophenyl)urea;
N-[4-(3-amino-1,2-benzisoxazol-4-yl)phenyl]-N′-(3,5-difluorophenyl)urea;
N-[4-(3-amino-1,2-benzisoxazol-4-yl)phenyl]-N′-(3-methoxyphenyl)urea;
N-[4-(3-amino-1,2-benzisoxazol-4-yl)phenyl]-N′-(4-methoxyphenyl)urea;
N-[4-(3-amino-1,2-benzisoxazol-4-yl)phenyl]urea;
N-[4-(3-amino-1,2-benzisoxazol-4-yl)phenyl]-N′-(3-nitrophenyl)urea;
N-[4-(3-amino-1,2-benzisoxazol-4-yl)phenyl]-N′-(4-fluorophenyl)urea;
N-[4-(3-amino-1,2-benzisoxazol-4-yl)phenyl]-N′-(2-fluorophenyl)urea;
N-[4-(3-amino-1,2-benzisoxazol-4-yl)phenyl]-N′-(3-chloro-4-fluorophenyl)urea;
N-[4-(3-amino-1,2-benzisoxazol-4-yl)phenyl]-N′-(3-chloro-4-methoxyphenyl)urea;
N-[4-(3-amino-1,2-benzisoxazol-4-yl)phenyl]-N′-[4-(dimethylamino)phenyl]urea;
N-[4-(3-amino-1,2-benzisoxazol-4-yl)phenyl]-N′-[4-(trifluoromethoxy)phenyl]urea;
N-[4-(3-amino-1,2-benzisoxazol-4-yl)phenyl]-N′-[2-(trifluoromethoxy)phenyl]urea;
N-[4-(3-amino-1,2-benzisoxazol-4-yl)phenyl]-N′-[3,5-bis(trifluoromethyl)phenyl]urea;
N-[4-(3-amino-1,2-benzisoxazol-4-yl)phenyl]-N′-(3-chloro-4-methylphenyl)urea;
N-[4-(3-amino-7-methoxy-1,2-benzisoxazol-4-yl)phenyl]-N′-[3,5-bis(trifluoromethyl)phenyl]urea;
N-[4-(3-amino-7-methoxy-1,2-benzisoxazol-4-yl)phenyl]-N′-[4-(trifluoromethoxy)phenyl]urea;
N-[4-(3-amino-7-methoxy-1,2-benzisoxazol-4-yl)phenyl]-N′-(3-fluorophenyl)urea;
N-[4-(3-amino-7-methoxy-1,2-benzisoxazol-4-yl)phenyl]-N′-(3-methoxyphenyl)urea;
N-[4-(3-amino-7-methoxy-1,2-benzisoxazol-4-yl)phenyl]-N′-(3,5-difluorophenyl)urea;
N-[4-(3-amino-7-methoxy-1,2-benzisoxazol-4-yl)phenyl]-N′-(4-methylphenyl)urea;
N-[4-(3-amino-7-methoxy-1,2-benzisoxazol-4-yl)phenyl]-N′-(3-bromophenyl)urea;
N-[4-(3-amino-7-methoxy-1,2-benzisoxazol-4-yl)phenyl]-N′-(3,5-dimethylphenyl)urea;
N-[4-(3-amino-7-methoxy-1,2-benzisoxazol-4-yl)phenyl]-N′-[4-(dimethylamino)phenyl]urea;
N-[4-(3-amino-7-methyl-1,2-benzisoxazol-4-yl)phenyl]-N′-(3-methylphenyl)urea;
N-[4-(3-amino-7-methyl-1,2-benzisoxazol-4-yl)phenyl]-N′-(3-chlorophenyl)urea;
N-[4-(3-amino-7-methyl-1,2-benzisoxazol-4-yl)phenyl]-N′-(2-fluoro-5-methylphenyl)urea;
N-[4-(3-amino-7-methyl-1,2-benzisoxazol-4-yl)phenyl]-N′-[2-fluoro-5-(trifluoromethyl)phenyl]urea;
N-[4-(3-amino-7-methyl-1,2-benzisoxazol-4-yl)phenyl]-N′-[3-(trifluoromethyl)phenyl]urea;
N-[4-(3-amino-7-methyl-1,2-benzisoxazol-4-yl)phenyl]-N′-(3,5-dimethylphenyl)urea;
N-[4-(3-amino-7-methyl-1,2-benzisoxazol-4-yl)phenyl]-N′-(3-ethylphenyl)urea;
N-[4-(3-amino-7-methyl-1,2-benzisoxazol-4-yl)phenyl]-N′-(4-methylphenyl)urea;
N-[4-(3-amino-7-methyl-1,2-benzisoxazol-4-yl)phenyl]-N′-[4-(trifluoromethoxy)phenyl]urea;
N-[4-(3-amino-7-methyl-1,2-benzisoxazol-4-yl)phenyl]-N′-(3-fluoro-4-methylphenyl)urea;
N-[4-(3-amino-7-methyl-1,2-benzisoxazol-4-yl)phenyl]-N′-(3-methoxyphenyl)urea;
N-[4-(3-amino-7-methyl-1,2-benzisoxazol-4-yl)phenyl]-N′-phenylurea;
N-[4-(3-amino-7-methyl-1,2-benzisoxazol-4-yl)phenyl]-N′-[3,5-bis(trifluoromethyl)phenyl]urea;
N-[4-(3-amino-7-methyl-1,2-benzisoxazol-4-yl)phenyl]-N′-(3-bromophenyl)urea;
N-[4-(3-amino-7-methyl-1,2-benzisoxazol-4-yl)phenyl]-N′-(3-fluorophenyl)urea;
N-[4-(3-amino-7-methoxy-1,2-benzisoxazol-4-yl)phenyl]-N′-[4-fluoro-3-(trifluoromethyl)phenyl]urea;
N-[4-(3-amino-7-methoxy-1,2-benzisoxazol-4-yl)phenyl]-N′-(4-fluoro-3-methylphenyl)urea;
N-[4-(3-amino-7-fluoro-1,2-benzisoxazol-4-yl)phenyl]-N′-[3-(trifluoromethyl)phenyl]urea;
N-[4-(3-amino-7-fluoro-1,2-benzisoxazol-4-yl)phenyl]-N′-(3-chlorophenyl)urea;
N-[4-(3-amino-7-fluoro-1,2-benzisoxazol-4-yl)phenyl]-N′-[4-fluoro-3-(trifluoromethyl)phenyl]urea;
N-[4-(3-amino-7-fluoro-1,2-benzisoxazol-4-yl)phenyl]-N′-(3-methylphenyl)urea;
N-[4-(3-amino-7-fluoro-1,2-benzisoxazol-4-yl)phenyl]-N′-[2-fluoro-5-(trifluoromethyl)phenyl]urea;
N-[4-(3-amino-7-fluoro-1,2-benzisoxazol-4-yl)phenyl]-N′-(2-fluoro-5-methylphenyl)urea;
N-{4-[3-amino-7-(trifluoromethoxy)-1,2-benzisoxazol-4-yl]phenyl}-N′-[2-fluoro-5-(trifluoromethyl)phenyl]urea;
N-{4-[3-amino-7-(trifluoromethoxy)-1,2-benzisoxazol-4-yl]phenyl}-N′-[3-(trifluoromethyl)phenyl]urea;
N-{4-[3-amino-7-(trifluoromethoxy)-1,2-benzisoxazol-4-yl]phenyl}-N′-(2-fluoro-5-methylphenyl)urea;
N-{4-[3-amino-7-(trifluoromethoxy)-1,2-benzisoxazol-4-yl]phenyl}-N′-(3-chlorophenyl)urea;
N-{4-[3-amino-7-(trifluoromethoxy)-1,2-benzisoxazol-4-yl]phenyl}-N′-(3-bromophenyl)urea;
N-{4-[3-amino-7-(trifluoromethoxy)-1,2-benzisoxazol-4-yl]phenyl}-N′-[4-fluoro-3-(trifluoromethyl)phenyl]urea;
N-[4-[3-amino-1H-indazol-4-yl]phenyl]-N′-(2-fluoro-5-methoylphenyl)urea;
N-{4-[3-amino-7-(trifluoromethoxy)-1,2-benzisoxazol-4-yl]phenyl}-N′-(4-fluoro-3-methylphenyl)urea;
N-[4-(3-amino-1H-indazol-4-yl)phenyl]-N′-(3-methylphenyl)urea;
N-[4-(3-amino-1H-indazol-4-yl)phenyl]-N′-(3,5-dimethoxyphenyl)urea;
N-[4-(3-amino-1H-indazol-4-yl)phenyl]-N′-(3-chlorophenyl)urea;
N-[4-(3-amino-1H-indazol-4-yl)phenyl]-N′-[3-(trifluoromethyl)phenyl]urea;
N-[4-(3-amino-1H-indazol-4-yl)phenyl]-N′-[2-fluoro-5-(trifluormethyl)phenyl]urea;
N-[4-(3-amino-1H-indazol-4-yl)phenyl]-N′-(3-bromophenyl)urea;
N-[4-(3-amino-1H-indazol-4-yl)phenyl]-N′-(3-bromo-4-methylphenyl)urea;
N-[4-(3-amino-1H-indazol-4-yl)phenyl]-N′-(3-ethylphenyl)urea;
N-[4-(3-amino-1H-indazol-4-yl)phenyl]-N′-phenyl urea;
N-[4-(3-amino-1H-indazol-4-yl)phenyl]-N′-(3-fluoro-4-methylphenyl)urea;
N-[4-(3-amino-1H-indazol-4-yl)phenyl]-N′-(2-fluorophenyl)urea;
N-[4-(3-amino-1H-indazol-4-yl)phenyl]-N′-(4-fluorophenyl)urea;
N-[4-(3-amino-1H-indazol-4-yl)phenyl]-N′-(3-fluorophenyl)urea;
N-[4-(3-amino-1H-indazol-4-yl)phenyl]-N′-(3-hydroxyphenyl)urea;
N-[4-(3-amino-1H-indazol-4-yl)phenyl]-N′-(3-methylphenyl)urea;
N-[4-(3-amino-1H-indazol-4-yl)-2-fluorophenyl]-N′-(2-fluoro-5-methylphenyl)urea;
N-[4-(3-amino-1H-indazol-4-yl)phenyl]-N′-[4-fluoro-3-(trifluoromethyl)phenyl]urea;
N-[4-(3-amino-1H-indazol-4-yl)phenyl]-N′-[2-fluoro-3-(trifluoromethyl)phenyl]urea;
N-[4-(3-amino-1H-indazol-4-yl)phenyl]-N′-(4-bromo-2-fluorophenyl)urea;
N-[4-(3-amino-1H-indazol-4-yl)phenyl]-N′-(5-fluoro-2-methylphenyl)urea;
N-[4-(3-amino-1H-indazol-4-yl)phenyl]-N′-(4-fluoro-3-methylphenyl)urea;
N-[4-(3-amino-1H-indazol-4-yl)phenyl]-N′-[2-fluoro-5-(hydroxymethyl)phenyl]urea;
3-[({[4-(3-amino-1H-indazol-4-yl)phenyl]amino}carbonyl)amino]-4-fluorobenzoic acid; and
Methyl 3-[({[4-(3-amino-1H-indazol-4-yl)phenyl]amino}carbonyl)amino]-4-fluorobenzoate.

55. The composition of claim 54, wherein the RTK inhibitor is N-[4-[3-amino-1H-indazol-4-yl]phenyl]-N′-(2-fluoro-5-methoylphenyl)urea.

56. The composition of claim 34, wherein the anti-inflammatory agent is selected from the group consisting of COXII inhibitor and glucocorticoids.

57. The composition of claim 55, wherein the anti-inflammatory agent is selected from the group consisting of COXII inhibitors, COX I/II inhibitors, and glucocorticoids.

58. The composition of claim 57, wherein the anti-inflammatory agent is a COXII inhibitor.

59. The composition of claim 58, wherein the COXII inhibitor is a 3-benzoylphenylacetic acid derivative.

60. The composition of claim 56, wherein the anti-inflammatory agent is a COX II inhibitor.

61. The composition of claim 60, wherein the COXII inhibitor is a 3-benzoylphenylacetic acid derivative.

62. The composition of claim 34, wherein the amount of RTK inhibitor in the composition is from 0.0001% to about 30%, and the amount of anti-inflammatory agent in the composition is from 0.01% to about 10%.

63. The composition of claim 34, wherein the composition is a suspension.

64. The composition of claim 34, wherein the composition is a gel.


SEQUENCE LISTING (4 sequences)

SEQ ID NO: 1 - DNA, 19 bp, Homo sapiens
ttactgctgtacctccacc

SEQ ID NO: 2 - DNA, 19 bp, homo sapiens
acaggacggcttgaagatg

SEQ ID NO: 3 - DNA, 20 bp, mouse
cactgcaaacggggaaatgg

SEQ ID NO: 4 - DNA, 20 bp, mouse
tgagatggactgtcggatgg